(19) United States
(12) Patent Application Publication
(10) Pub. No.: US 2006/0025768 A1
(43) Pub. Date: Feb. 2, 2006

(54) Top loading spinal fixation device and instruments for loading and handling the same

(76) Inventors: Andy Iott, West Chester, PA (US); Larry Binder, Langhorne, PA (US); Brian Dec, Lafayette Hill, PA (US); Kelly Baker, Coatesville, PA (US)

Correspondence Address: JONES DAY, 222 EAST 41ST STREET, NEW YORK, NY, 10017-6702, US

(21) Appl. No.: 10/886,392

(22) Filed: Jul. 6, 2004

Related U.S. Application Data
(60) Provisional application No. 60/484,286, filed on Jul. 3, 2003.

(51) Int. Cl. A61F 2/30

(52) U.S. Cl. 606/61

(57) ABSTRACT

A system for introducing an elongated spinal rod comprising a bone fastener having a head, the head including a top end, a bottom end, and a pair of side walls connected at the bottom end defining an elongated U-shaped channel having a first longitudinal axis and a top opening located at the top end. The top opening and the channel further define a passageway configured for receiving the spinal rod and each side wall further defining a first end face and a second end face, the second end face opposite the first end face. A bone engaging element extends from the bottom end of the head for anchoring the bone fastener to the bone. The system further comprises a tool for introducing the rod into the U-shaped channel. The tool includes a first member having a distal end and a proximal end. The distal end is configured for guiding the rod through the passageway to introduce the rod into the U-shaped channel through the top opening. A second member defines a second longitudinal axis substantially perpendicular to the first longitudinal axis. The second member has first and second ends, the second end having a pair of jaws for securing the tool to one of the side walls of the head of the bone fastener such that second member is disposed outside of the passageway of the spinal rod, and preferably so as not to interfere with or block access to the top opening. The first member is operably associated with the second member such that the first member is pivotable with respect to the second member and linearly translatable in a direction parallel to the second longitudinal axis between the first and second ends of the second member along the second longitudinal axis for introducing the spinal rod through the top opening into the passageway.

### FIELD OF THE INVENTION

The present invention is directed to spinal fixation devices, more specifically, to bone screws having a rod receiving channel and the instruments for introducing spinal rods into the channel.

### BACKGROUND OF THE INVENTION

To correct spinal deformities caused by either injury or defects in the natural formation of the spine, a spinal fixation system is typically employed in an osteosynthesis surgical procedure. A typical spinal fixation system may incorporate a plurality of bone fasteners substantially aligned along the length of the spine each interconnected by a spinal rod. The spinal rod is received in the channels formed in the head or body of the bone fasteners. The bone fastener may anchor to the lamina or pedicle of the vertebral body by either a threaded shank or hook which extends from the head or body of the fastener. The hook-type bone fastener may, in addition, be anchored to the transverse process. Typically, with the bone fasteners rigidly fixed to the vertebral bodies of the spinal column, the rod receiving channels of the heads are not aligned. This misalignment may require that the spinal rod be bent so as to properly seat within the rod receiving channel. The spinal rod may additionally be bent to provide a selected configuration for correction of the spinal defect. The spinal rod may also have a straight configuration. The forced interconnection of the bone fastener via the bent spinal rod transfers corrective stresses to the patient's spine.

A fastener often used in the internal fixation system, and contemplated for use with respect to the instruments according to the present invention, is referred to as the top loading fastener. More specifically, the bone fastener may include a head or body having a U-shaped channel therethrough wherein the opening of the channel is atop the head of the bone fastener. The U-shaped channel defines a passageway for receipt of the spinal rod and the top opening receives a locking cap to secure the rod in the U-shaped channel. The head of the bone fastener itself is typically spherical or cylindrical in nature in which the external side surfaces of the wall are arcuate in shape.

Implanting an internal spine fixation system requires instrumentation for handling and manipulating top loading bone fasteners as described. In addition, instruments for installing the spinal rod into the U-shaped channel of an anchored fastener are also necessary. In the case of rod introduction instrumentation, more than one tool is often used wherein one tool attaches to the bone fastener to either stabilize the head of the fastener during rod installation or to act as a guide to the second instrument that installs the spinal rod. A problem with an installation procedure requiring two tools is that does not enable the surgeon to maintain one hand free, possibly requiring multiple surgeons to complete the procedure. Where a single instrument is provided for both stabilizing the implant and applying an introduction force to the spinal rod, the instrument typically engages the head of the bone fastener and straddles the U-shaped channel. Generally the instrument attaches in a manner that encroaches over the top opening of the U-shaped channel, severely limiting access to the open channel. In addition, a rod installing instrument which engages the head on both sides of the U-shaped channel is limited in its angle of approach with respect to the fastener. The surgical area may be reduced by the natural anatomy of the patient and an instrument that must be secured to both lateral sides of the U-shaped channel in the head of the fastener further reduces the ability of the surgeon to select the manner of approach when installing the rod.

Similar problems exist with respect to instruments that are used solely in handling and manipulation of top loading bone fasteners. Hand instruments that require engagement with both lateral sides of the U-shaped channel of the head of the bone fastener at the same time may complicate the installation process especially where once again the geometry of the surgical area does not allow the surgeon to grasp both lateral sides of the head and install the fastener.

Instrumentation is needed that will adequately engage a single lateral wall of a head of a bone fastener for secure handling and manipulation. Where applicable, a single instrument capable of securing about the single lateral side wall and driving a spinal rod into its receiving channel would be advantageous. It is desirable for the instrument to be adequately configured for secure engagement with the arcuate side surfaces of the lateral walls. An instrument capable of affixing to a single lateral wall is additionally advantageous because its use permits the surgeon to selectively approach the fastener from either side of the U-shaped channel.

### SUMMARY OF THE INVENTION

The present invention in one embodiment is directed to a bone fastener for correcting spinal deformities, the bone fastener comprises a head having a top surface, a bottom surface, and first and second side walls. The first and second side walls each have a first end face and a second end face, the second end face opposite the first end face. The first and second side walls define an opening in the top surface and a U-shaped channel through the head extending from the first end face to the second end face. The U-shaped channel has a longitudinal axis defining a passageway for receiving a spinal rod. The bone fastener further comprises a bone engaging element for anchoring to bone, the bone engaging element has a central axis and extends from the bottom of the head.

Each of the first and second end faces of the first and second side walls may include a recess configured for mating engagement with an instrument for handling and manipulating the bone fastener and spinal rod such that the instrument is located outside of the passageway, preferably in a manner that does not interfere with or block access to the passageway or the top opening. Each of the recesses may be configured to be in communication with the U-shaped channel, and may include a seat for secure engagement with the instrument. The seats may define an angle with respect to the longitudinal axis of the U-shaped channel. The angle may range, for example, from about 15° to about 90°, or preferably from about 45° to about 75°, and more preferably from about 55° to about 60°. Each of the recesses of the first and second end faces may further define a first end wall and a second end wall, the seat located therebetween, wherein the first and second end walls are dimensioned and configured for secure engagement with the instrument thereby preventing the fastener from pivoting with respect to the instrument. In addition, the dimensions and configurations of the seat and first and second end walls may facilitate a mated engagement with the instrument.

The first and second side walls of the bone fastener may be substantially arcuate. Each of the first and second side walls may define an interior surface and an exterior side surface, in which the internal surface further defines and communicates with the U-shaped channel and the exterior side surface is located opposite the internal surface. The exterior side surfaces of the first and second side walls may include a cavity configured for engaging another instrument for handling the bone fastener and spinal rod. The cavity may vary in depth in a direction going from the top surface to the bottom surface. The cavity may also define a shoulder for engaging the instrument so as to apply a force in a direction along the central axis of the bone engaging element. The internal surface of the first and second side walls may be threaded for engaging an externally threaded cap to retain the spinal rod in the U-shaped channel.

The bone engaging element of the bone fastener may be integral with the head of the bone fastener. The bone engaging element may be either a threaded shank or a hook. Alternatively, the bone engaging element may be separably engaged with the bottom surface of the head, the bone engaging element having a proximal end and a distal end, the distal end being configured for anchoring to the bone. The head of the bone fastener may further include an internal cavity configured for securely receiving the proximal end of the bone engaging element such that the bone engaging element lies at a selected angle relative to the head.

The present invention is also directed to a system for introducing an elongated spinal rod. The system comprises a bone fastener having a head, the head including a top surface, a bottom surface, a pair of side walls defining an elongated U-shaped channel having a first longitudinal axis and a top opening located at the top surface, the top opening and the channel further defining a passageway configured for receiving the spinal rod, each side wall further defining a first end face and a second end face, the second end face opposite the first end face, and a bone engaging element extending from the bottom surface of the head for anchoring the bone fastener to the bone. The system further comprising a tool for introducing the rod into the U-shaped channel. The tool includes a first member having a distal end and a proximal end, the distal end configured for guiding the rod through the passageway to introduce the rod into the U-shaped channel through the top opening. The tool also includes a second member defining a second longitudinal axis substantially perpendicular to the first longitudinal axis, the second member having first and second ends, the second end having a pair of jaws for securing the tool to one of the side walls of the head of the bone fastener such that second member is disposed substantially outside of the passageway of the spinal rod, preferably in a manner that does not interfere with or block access to the U-shaped channel, the passageway or the top opening. The first member is operably associated with the second member such that the first member is pivotable with respect to the second member and linearly translatable in a direction parallel to the second longitudinal axis between the first and second ends of the second member along the second longitudinal axis for introducing the spinal rod through the top opening into the passageway.

Wherein the first and second end faces of the bone fastener each define a recess, the jaws of the tool may include a first extension and a second extension, the second extension located opposite the first extension, wherein further the first extension is configured to engage the recess of the end face and the second extension is configured to engage the recess of the second end face to fix the instrument with respect to the fastener. Each of the recesses of first and second end faces may further define a seat and two opposed end walls dimensioned and configured for mating engagement with one of the first and second extensions. Each of the seats may form an angle with respect to the longitudinal axis of the U-shaped channel. The angle may range, for example, from about 15° to about 90°, or preferably from about 45° to about 75°, and more preferably from about 55° to about 60°. The first and second extension may be dimensioned and configured so as to form a secure fit within the recesses upon engagement with the sidewalls thereby preventing rotation of the bone fastener with respect to the jaws in at least one direction.

Wherein the side walls further define an exterior side surface and the jaws define an interior surface, the interior surface may be configured for mating engagement with the exterior side surface of the side walls so as to prevent rotation of the bone fastener with respect to the instrument. The exterior side surfaces may be substantially arcuate, and the interior surface of the jaws may be substantially arcuate and configured for secure engagement with the exterior side surface. In addition the interior surface may be configured for a mated engagement with the exterior side surface.

The present invention is also directed, in one embodiment, to an instrument for introducing an elongated spinal rod into a U-shaped channel of a bone fastener having a top opening, the bone fastener having a pair of side walls defining the U-shaped channel and the top opening to the U-shaped channel, the top opening and the U-shaped channel further defining a passageway for the spinal rod in the direction of elongation. The instrument comprises a first member having a longitudinal axis, a proximal end and a distal end configured for engaging one of the side walls to secure the instrument to the fastener such that the instrument is disposed outside of the passageway of the spinal rod, preferably in a manner that does not interfere with or block access to the U-shaped channel, the passageway or the top opening. The instrument also comprises a second member having a first end and a second end, the second end configured for guiding the spinal rod through the passageway and into the U-shaped channel. The second member is operably associated with the first member such that the second end of the second member is pivotable with respect to the distal end of the first member and the second member is linearly translatable along the longitudinal axis with respect to the first member for introducing the spinal rod through the passageway and into the U-shaped channel.

The side walls of the bone fastener may include a first and second end face defining a recess and, the distal end of the first member may include a pair of jaws, the jaws having a first extension and a second extension, the second extension located opposite the first extension, wherein the first extension may be configured to engage the recess of the first end face and the second extension may be configured to engage the recess of the second end face. The instrument may further comprise a shaft or housing having a through bore or channel, the first member received in the channel, the housing linearly translatable along the longitudinal axis of the first member, and the second member pivotally mounted to the housing such that the second end of the second member is pivotable and linearly translatable with respect to the distal end of the first member.

The first member may have an external surface at least a portion of which has threads or gear teeth, and the instrument may further comprise an assembly comprising a pinion, a knob, and a pawl or switch. The pinion and switch threadably engaged with the gear teeth of the first member and operably associated with the second member for translating the second member with respect to the first member upon rotation of the knob. The switch may be configured to toggle between a first position limiting translation of the second member relative to the first member in a first direction and a second position where the second member is limited to translation in a second direction opposite the first direction. In addition, the switch may have a third position to prevent translation of the second member relative to the first member, and the switch may have a fourth position where the second member may translate in either direction along the first member.

The first member may also comprise a shaft having a central through channel defining a chamber at the distal end of the first member and an elongated member having a proximal end and a distal end received in the channel of the first member. The jaws may depend from the distal end of the elongated member and at least a portion of the jaws may be located in the chamber.

The instrument may further comprise a cam lock associated with the proximal end of the elongated member for securing the elongated member in the central bore of the first member. Rotation of the cam lock may preferably translate the elongated member linearly with respect to the first member such that the jaws engage the chamber to close the jaws.

The second member of the instrument may include a pair of arms associated with the second end and configured for guiding the rod through the passageway and into the U-shaped channel. The arms may be preferably angled with respect to the second member. In addition, the arms may be spaced relative to one another such that the head of the bone fastener may be accommodated between the arms.

The instrument may further comprise a pair of pivotally connected handles. The second member may be fixed with respect to one handle, and the first member operably associated with the other handle such that the second end of the second member is pivotable with respect to the distal end of the first member. The second member further comprises a ratchet mechanism associated with the pair of articulating handles. The ratchet mechanism may be configured to selectively pivotally position the second end of the second member in a fixed relationship with respect to the distal end of the first member.

In another embodiment of an instrument for introducing an elongated spinal rod into a U-shaped channel of a bone fastener having a top opening, the instrument comprises a first member including a shaft, at least a portion of which has threads, the shaft having a longitudinal axis, a distal end configured for guiding the rod, a proximal end, and a knob associated with the proximal end. Alternatively, at least a portion of the first member may include external gear teeth. The first member and knob may also form a continuous through bore or channel extending from the proximal end to the distal end for receiving therein additional surgical instruments, for example a tool to secure a locking cap to secure the rod into the U-shaped channel. The rod introduction instrument further comprises a sleeve defining a longitudinal axis and having a through bore, the first member received in the through bore. In addition, the instrument comprises a second member having a first end and a second end, wherein the first end is configured to engage one of the side walls to secure the instrument to the fastener such that the instrument is disposed outside the passageway of the spinal rod defined by the U-shaped channel, preferably in a manner that does not interfere with or block access to the U-shaped channel, the passageway or the top opening. The second member is pivotally engaged with the sleeve for positioning the distal end of the first member with respect to the first end of the second member. Rotation of the knob causes the distal end of the first member to move linearly along the direction of the longitudinal axis of the first member with respect to the sleeve for introducing the spinal rod through the passageway and into the U-shaped channel.

Each of the side walls of the bone fastener may include a first and second end face, defining a recess, and the first end of the second member includes a pair of jaws, the jaws having a first extension and a second extension, the second extension located opposite the first extension, wherein further the first extension is configured to engage the recess of the first end face of one of the side walls and the second extension is configured to engage the recess of the second end face of the side wall. The first and second extension are dimensioned and configured so as to form a secure engagement with the opposing end walls and the seat to prevent rotation of the fastener with respect to the jaws.

The second end of the second member of the instrument may include a pair of pivoting arms for bringing the first end of the second member into engagement with the side wall of the bone fastener. The second end of the second member may include a screw and a nut threadably disposed about the screw for selectively fixing the distance between the pair of arms. The second member may also include two parallel brackets disposed between the first end and the second end, wherein the brackets are configured for pivotal engagement with the sleeve, the sleeve disposed between the brackets such that the distal end of the first member is pivotable with respect to the first end of the second member.

The first member includes a rod engaging tip disposed about the distal end of the first member for guiding the rod, the rod engaging tip having a threaded central chamber for threaded engagement with the threaded portion of the first member and configured so as to be non-rotatable with respect to the sleeve, such that rotation of the first member causes the rod engaging tip to move linearly with respect to the sleeve and the first member. Alternatively, the engagement between the first member, the rod engaging tip and sleeve may be formed by a rack and pinion configuration for providing linear translation of the rod engaging tip with respect to the sleeve. The rod engaging tip may include two parallel arches configured for secure engagement about the rod. Rotation of the knob may also cause the rod engaging tip to move with respect to the first end of the second member.

The present invention is also directed to a system, for example a kit, for introducing an elongated spinal rod, the system comprising a tool for introducing the spinal rod into the U-shaped channel of a bone fastener having two side walls defining the U-shaped channel and including exterior side-surfaces having cavities configured for engagement with the tool. The tool comprises a shaft having a distal end and a proximal end, a handle associated with the proximal end, and a prong associated with the distal end. The prong has a first leg and a second leg, in which the second leg is spaced from and parallel to the first leg. The first and second leg are dimensioned and configured to accommodate the head with the spinal rod proximate the top opening of the bone fastener in between the first and second leg. The handle being substantially aligned with the passageway of the spinal rod. The first and second legs each include an extension, the extensions are dimensioned and configured for cammed engagement with the cavity of the exterior side surface so as to apply a pivoting force to the rod to introduce the rod into the U-shaped channel.

In another embodiment of an instrument according to the present invention for manipulating a bone fastener having a pair of side walls defining a U-shaped channel having a top opening, the U-shaped channel and top opening defining a passageway configured for receiving a spinal rod, the instrument comprising an elongated member having a distal end and a proximal end; the distal end defining a pair of jaws configured for securing about one of the side walls of the fastener such that the instrument is disposed substantially outside the passageway, preferably in a manner that does not interfere with or block access to the U-shaped channel, the passageway or the top opening. The instrument also includes a handle associated with the proximal end and a shaft having a through bore. The shaft is associated with the distal end of the elongated member and has a first position where the jaws are resiliently biased open and a second position where the jaws are drawn together in a closed position to secure about the side wall of the head of the fastener. Wherein each of the side walls include a first and second end face, each first and second end face defining a recess, the jaws may have a first extension and a second extension, the second extension located opposite the first extension, wherein further the first extension is configured to engage the recess of the first end face and the second extension is configured to engage the recess of the second end face. The shaft may slide with respect to the elongated member and at least a portion of the jaws may be received in the through bore of the shaft to draw the jaws together in a closed position when the shaft is in the second position.

In an alternative embodiment of an instrument for manipulating a bone fastener having an enlarged head including a pair of side walls defining a U-shaped channel having a top opening, the U-shaped channel and top opening defining a passageway configured for receiving a spinal rod, the instrument comprises an elongated member having a distal end and a proximal end. The distal end defines a pair of pivotable jaws configured for securing about one of the side walls of the fastener such that the instrument is disposed substantially outside the passageway, preferably in a manner that does not interfere with or block access to the U-shaped channel, the passageway or the top opening. The proximal end has a pair of forceps handles for pivoting the jaws. The forceps handles have a first position where the jaws are configured in a closed position to secure about the side wall and at least a second position where the jaws are configured in an open position. Each of the side walls may include a first and second end face, each first and second end face defines a recess, and the jaws may have a first extension and a second extension, the second extension located opposite the first extension, wherein the first extension is configured to engage the recess of the first end face and the second extension is configured to engage the recess of the second end face. A ratchet mechanism is associated with the forceps handles, the ratchet mechanism configured such that the forceps handles may be selectively and fixedly positioned in the first or at least second position.

Another embodiment includes an instrument for manipulating a bone fastener having a pair of side walls defining a U-shaped channel having a top opening, the U-shaped channel and top opening defining a passageway configured for receiving a spinal rod. Each of the side walls include an exterior side surface having a cavity on the exterior side surface. The instrument comprises an elongated member having a distal end and a proximal end. The distal end defines a pair of pivotable jaws configured for securing to the pair of side walls of the fastener such that the instrument is substantially aligned with the passageway. The jaws define an interior surface including a first extension and a second extension each dimensioned and configured for pivotable engagement with the cavity, the second extension opposite the first extension. The proximal end has a pair of forceps handles for pivoting the jaws, the forceps handles have a first position where the jaws are configured in a closed position such that the first extension is engaged with the cavity of one of the side walls and the second extension is engaged with the cavity of the other side wall, and at least a second position where the jaws are configured in an open position. The proximal end may be substantially arcuate with respect to a portion of the elongated member located between the proximal and distal end. In addition, the interior surface of the jaws may define a chamber configured for secure engagement with at least a portion of the exterior side surfaces of the side walls of the bone fastener, in which the chamber is substantially circular or cylindrical.

### BRIEF DESCRIPTION OF THE DRAWINGS

The detailed description will be better understood in conjunction with the accompanying drawings, wherein like reference characters represent like elements, as follows:

FIG. 1 is plan view of a first illustrative embodiment of an instrument for a top loading device according to one aspect of the present invention, shown attached to a top loading spinal fixation device;

FIG. 2a is a perspective view of the spinal fixation device of FIG. 1 shown as a bone fastener in the form of a bone screw according to one aspect of the present invention;

FIG. 2b is a second illustrative embodiment of a bone fastener in the form of a bone hook according to one aspect of the present invention;

FIG. 2c is a partial perspective view of the bone fastener of FIG. 2a;

FIG. 2d is a top view of the bone fastener of FIG. 2a, taken along the line IId-IId of FIG. 2c;

FIG. 2e is a cross-sectional view of the head of bone fastener in FIG. 2a, taken along the line IId-IId of FIG. 2d;

FIG. 2f is an illustrative embodiment of a retaining cap for use with the bone fastener of FIG. 2a;

FIG. 2g is a partial plan view of the bone fastener of FIG. 2a;

FIG. 3a is a perspective view of the instrument of FIG. 1 engaged with the bone fastener of FIG. 2a;

FIG. 3b is a partial plan view of the instrument of FIG. 1 engaged with the bone fastener of FIG. 2a;

FIG. 3c is an exploded view of an illustrative embodiment of a first member of the instrument of FIG. 1 according to the present invention;

FIG. 3d is a side view of the first member of FIG. 3f, taken along the line IIId-IIId in FIG. 3c;

FIG. 3e is a perspective assembled view of the illustrative embodiment of the first member in FIG. 3c

FIG. 3f is a partial top view of the jaws of the instrument of FIG. 1 engaged with the bone fastener of FIG. 2a;

FIG. 3g is an illustrative embodiment of the jaws of the instrument of FIG. 1;

FIG. 3h is a side view of the jaws of FIG. 3g;

FIG. 3i is an exploded perspective view of the instrument of FIG. 1;

FIG. 5 is a perspective view of another illustrative embodiment of an instrument according to an aspect of the present invention;

FIG. 5a is an illustrative assembled embodiment of the second member and shaft of the instrument of FIG. 5;

FIG. 5b is a cross-sectional view of the second member of the instrument of FIG. 5, taken along the line Vb-Vb in FIG. 5a;

FIG. 5c is a plan view of an illustrative embodiment of a rod engaging tip of second member of FIG. 5a according to one aspect of the invention;

FIG. 5d is a plan view of the rod engaging tip of FIG. 5c taken along line Vd-Vd in FIG. 5c;

FIG. 5e is a top view of the instrument of FIG. 5;

FIG. 5f is a side view of the instrument in FIG. 7a;

FIG. 6a is a side view of another illustrative embodiment of an instrument according to an aspect of the present invention;

FIG. 6b is a top and partially cross-sectional view of the instrument of FIG. 6a, taken along the line VIb-VIb in FIG. 6a;

FIG. 6c is a plan view of the elongated member of the instrument of FIG. 6a;

FIG. 6d is a partial side view of the elongated member of FIG. 6c;

FIG. 6e is a perspective view of the jaws and shaft of the instrument of FIG. 6a;

FIG. 6f is a plan view of the shaft of the instrument of FIG. 6a;

FIG. 6g is a cross-sectional view of the shaft of the instrument of FIG. 6a, taken along the line VIg-VIg in FIG. 6f;

FIG. 7a is a perspective view of another illustrative embodiment of an instrument according to an aspect of the present invention;

FIG. 7b is a partial perspective view of the instrument of FIG. 7a engaged with the bone fastener of FIG. 2b;

FIG. 8a is perspective view of another illustrative embodiment of an instrument according to an aspect of the invention;

FIG. 8b is a partial perspective view of the instrument of FIG. 8a engaged with the bone fastener of FIG. 2b;

FIG. 9a is a perspective view of yet another illustrative embodiment of an instrument according to the present invention;

FIG. 9b is a side view of a the instrument of FIG. 9a;

FIG. 9c is a partial perspective view of the instrument of FIG. 9a engaged with the bone fastener of FIG. 9c;

FIG. 10a is a side view of another illustrative embodiment of an instrument according to an aspect of the present invention;

FIG. 10b is partial plan view of the prong of the instrument of FIG. 10a; and

FIG. 10c is a partial perspective view of the instrument of FIG. 10a engaged with the bone fastener of FIG. 2a and a spinal rod.

### DETAILED DESCRIPTION OF THE PREFERRED EMBODIMENTS

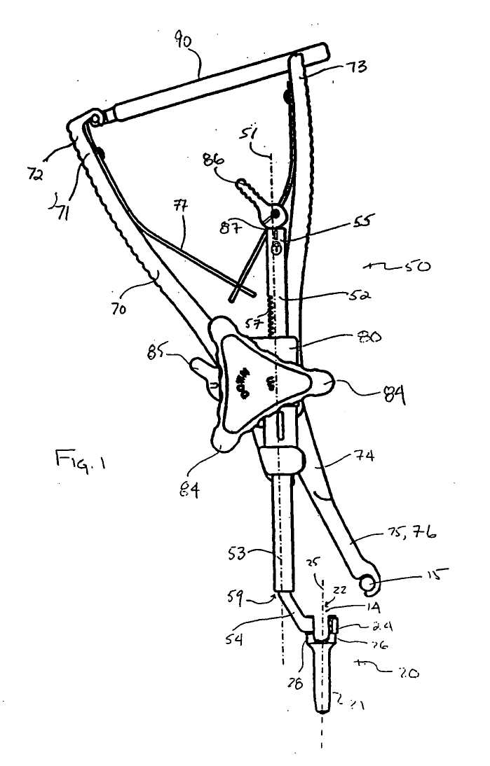

Referring to FIG. 1-12c there is shown a system for correction of spinal deformities including a bone fastener configured to fasten to the spine and having a U-shaped channel for receiving a longitudinal spinal rod. Several instruments are included in the system for handling and manipulating the bone fastener, as well as to manipulate and move the longitudinal spinal rod into the channel.

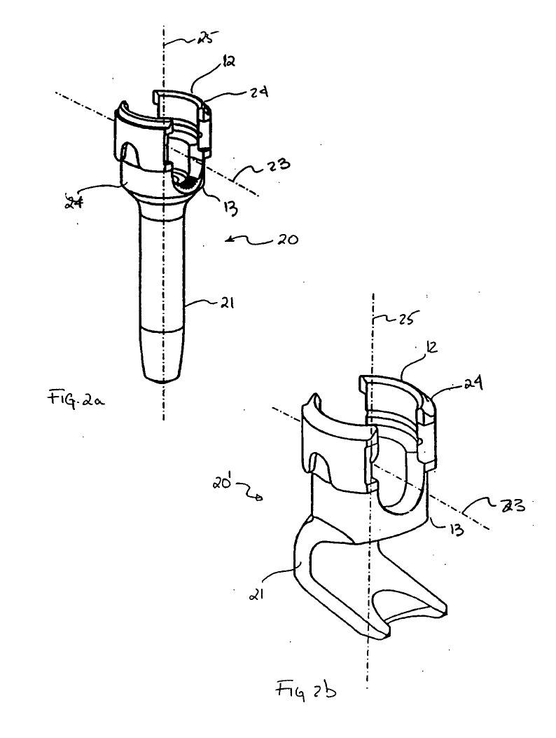

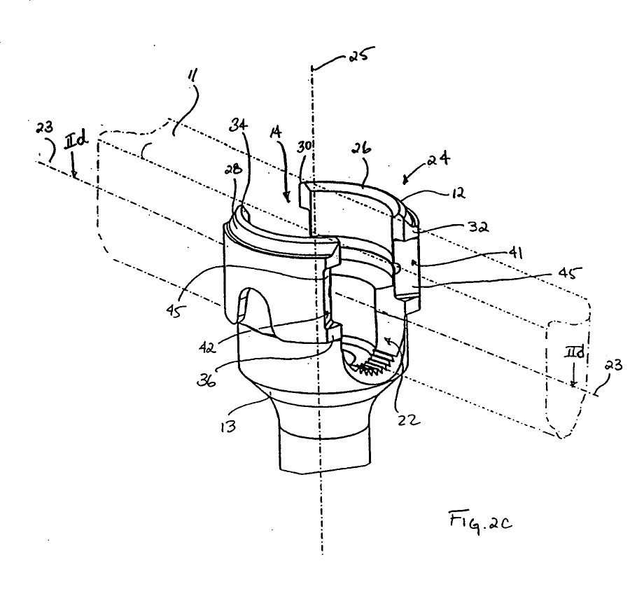

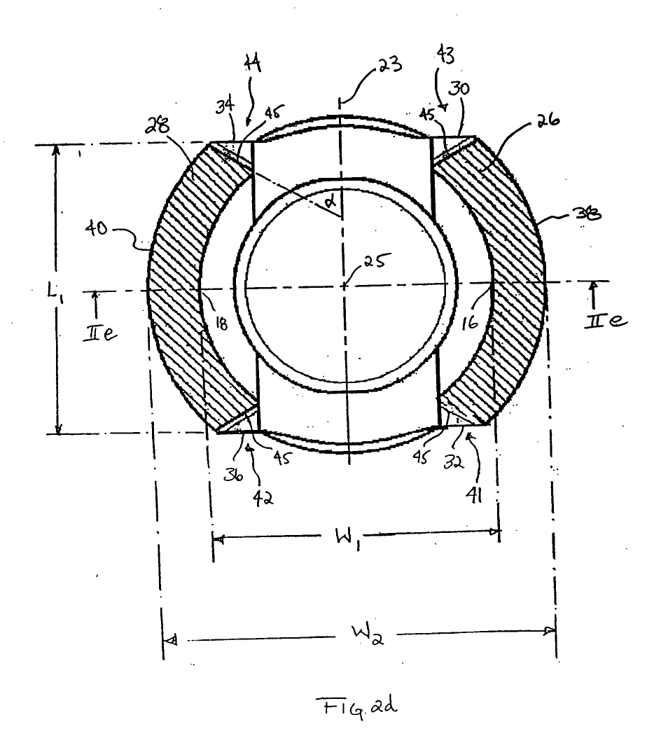

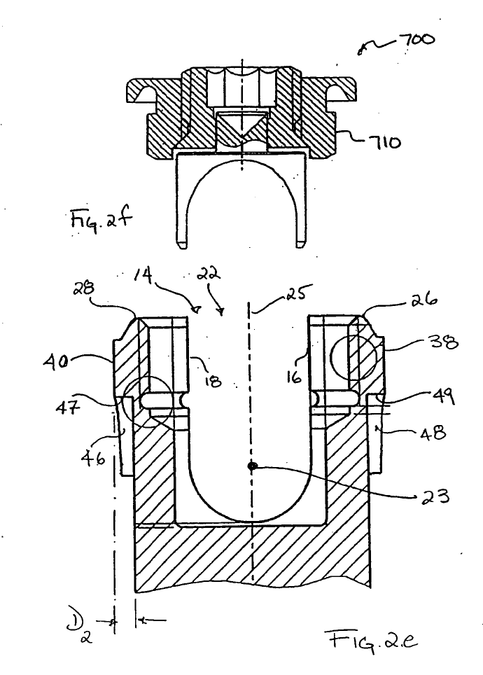

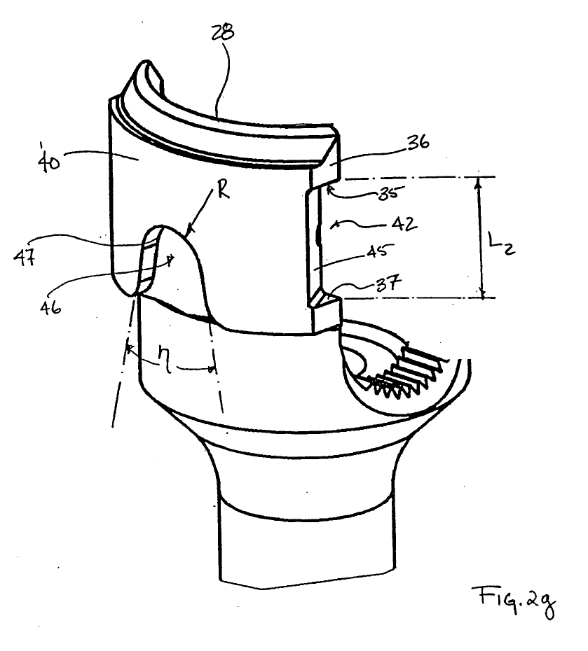

FIGS. 2a, 2b are first and second illustrative embodiments of bone fasteners 20, 20′ each having an enlarged head or body 24 and a bone engaging element 21, which defines a central axis 25. Shown in FIG. 2c is an enlarged view of the head 24, which includes a top surface 12, a bottom surface 13 and two lateral side walls 26, 28 defining a U-shaped channel 22 and a top opening 14 in the U-shaped channel 22. In the cross-section view of FIG. 2c, U-shaped channel 22 defines a longitudinal axis 23 normal to central axis 25. As will be discussed in greater detail below, U-shaped channel 22 is configured for receipt of a spinal rod 15. The spinal rod 15 is introduced through the top opening 14 for top loading of the U-shaped channel 22. Shown in FIG. 2c is the U-shaped channel 22 and top opening 14 thereby define a passageway 11 for spinal rod 15. The passageway 11 is shown as a substantially U-shaped volume through which the spinal rod may move prior to and upon being fixed in the U-shaped channel 22.

Referring now to FIG. 2e, U-shaped channel 22 is more specifically defined by internal surfaces 16, 18 of side walls 26, 28. As shown in FIG. 2d, internal surfaces 16, 18 may be substantially arcuate thereby defining a maximum span W₁ between internal surfaces 16, 18 of approximately 8 mm, although other dimensions for span W₁ are contemplated. The internal surfaces 16, 18 may include an internal thread, preferably a buttress thread as is well known in the art, for engaging an external thread 710 of cap 700, for example, shown in FIG. 2f, for retaining the spinal rod 15 in U-shaped channel 22, of course other structures and ways of attaching cap 700 to bone fastener 20 are contemplated and well known in the art. Referring back to FIG. 2d, side walls 26, 28 each include opposing end faces 30, 32 and 34, 36, respectively, and side walls 26, 28 each additionally defines external side surfaces 38, 40 respectively. The maximum width W₂ of the enlarged head 24, measured from external side surface 38, to the opposing external side surface 40, is approximately 12 mm, although other dimensions for width W₂ are contemplated. An advantageous aspect of the bone fastener 20 is its overall minimized width. Contributing to the overall reduced width is the fact that the engagement of spinal rod 15 and retaining cap 700 is generally contained within the U-shaped channel 22 of bone fastener 20.

Shown in FIG. 2c are end faces 32, 36 of side walls 26, 28 which may respectively further include recesses 41, 42 for engagement with an instrument for either handling and manipulation of the bone fastener 20 or introducing the spinal rod 15 into U-shaped channel 22. End faces 30, 36 are similarly configured having recesses 43, 44. End faces 30, 32, 34 and 36 form an angle with respect to the longitudinal axis 23 of about 85° to about 95° and preferably about 90°. Recesses 41, 42, 43, 44 may include a seat 45 cut out of or chamfered from end faces 30, 32, 34, 36 and configured so as to directly engage a handling or rod introducing instrument, as will be discussed in greater detail below. The seat 45 may be substantially flat, however other configurations are possible. For example, seat 45 may include a surface treatment to secure the engagement between the instrument and the recess. Alternate configurations are possible so as to accordingly match and mate with the handling and spinal introduction instruments of the system, which will be explained in greater detail below.

Referring again to FIG. 2d, end faces 30, 32, 34, 36 having respective recesses 43, 41, 44, 42, are each shown with seat 45 for secure engagement with an instrument for handling bone fastener 20 or introducing spinal rod 15 into the U-shaped channel 22. Recesses 41, 42, 43, 44 may be configured to communicate with U-shaped channel 22. Seat 45 may be cut inward toward the U-Shaped channel 22, thereby forming an angle α measured with respect to longitudinal axis 23 of U-shaped channel 22. An exemplary angle α may range from about 15° to about 90°, or angle α preferably may range from an angle of about 45° to about 75°, and more preferably range from about 55° to about 60°. The length L₁ measured from end face 30, 36 to end face 32, 34 is preferably about 10 mm, although other lengths L₁ are contemplated. This length L₁, while limiting the overall length of head 24, further locates recesses 41, 42, respectively, relative to recesses 43, 44. Additionally, shown in FIG. 2g with respect to side wall 28, recess 42 further defines end walls 35, 37 with seat 45 disposed therebetween. Recesses 41, 42 and 43 are similarly configured. End walls 35, 37 are configured and dimensioned to engage the instrument used for manipulating bone fastener 20 and introducing spinal rod 15 into the U-shaped channel 22, thereby providing additional surface contact between the instrument and the bone fastener 20 to secure the bone fastener 20 with respect to the instrument. The distance L₂ between the end walls 35, 37 is preferably about 5 mm. The distances L₁, L₂ and angle α assist, as will be explained in greater detail below, in achieving engagement between the handling and spinal rod introduction instruments and the bone fastener 20.

FIGS. 2c and 2d show external side walls 38 and 40 as being arcuate in shape, however additional configurations are possible, e.g., planar, etc. Additionally, shown in FIGS. 2e and 2g, external side surfaces 38, 40 may further define, respectively, recesses or cavities 48, 46 configured for engaging instruments for handling and manipulating the bone fastener 20 and introducing spinal rod 15. In a preferred embodiment, side walls 38, 40 includes arched-shaped recesses or cavities 46, 48. More clearly shown in FIG. 2d, the transition from external side surface 38, 40 to recess 46, 48 further defines shoulders 47, 49. Recesses 46, 48 may be generally configured for engaging instruments for manipulating and handling bone fastener 20, 20′. The depth D₁ of shoulders 47, 49 is dimensioned so as to provide sufficient material to which an instrument may securely engage the head 24 for handling or engaging the bone fastener 20. The depth D₁ of recesses 46, 48 may taper or vary in a direction going from the top surface 12 to the bottom surface 13 in which the depth D₁ may range from about 0.5 mm to about 1.5 mm, and may preferably be about 1 mm. Now referring to FIG. 2g, as shown with respect to side wall 28 the contour of recesses 46, 48 may generally define an arch-like or arcuate shape. The top of the arch of recess 46, 48 may be defined by a radius of curvature R which, for example, may be about 1 mm to about 2 mm, and preferably may be about 1.5 mm, and the bottom of the arch of recesses 46, 48 may span to define an included angle η of, for example, about 15° to about 30° and preferably about 20°. However, it is understood that other configurations and dimensions are possible, including different radius of curvature R and included angle η, such that surgical instruments may engage recesses 46, 48 for the purpose of manipulating and handling bone fastener 20, 20′.

Shown in FIG. 2a extending from the bottom surface 13 and integral with enlarged head 24 of bone fastener 20 is a bone engaging element in the form of a threaded shaft or shank 21 for anchoring bone fastener 20 to bone. However, other configurations are possible, for example as shown in FIG. 2b, bone fastener 20 includes a hook which may extend from head 24. In an alternative embodiment of bone fastener 20, the threaded shaft or hook 21 may be separably extendable from the body or head 24, thereby forming a poly-axial bone fastener (not shown). In a poly-axial embodiment, the head or body 24 may be further configured to include an internal cavity for receiving therein the separable bone engaging element. The internal cavity may be semi-spherical in shape. The separable threaded shaft or hook 21 may include an enlarged semi-spherical top that is captured in and poly-axially rotatable with respect to the internal semi-spherical cavity in the body 24. The bone engaging element may poly-axially rotate within the body until the bone engaging element is fixed or locked in the body. The body 24 of the poly-axial embodiment, may be otherwise substantially the same as the head 24 previously described herein.

Referring again to FIG. 1, a first illustrative embodiment of a rod introduction instrument is shown as instrument 50. Spinal rod introducing instrument 50 engages, as shown, bone fastener 20 for introducing spinal rod 15 into the elongated U-shaped channel 22 of the head or body 24 of the bone fastener. Instrument 50 may similarly engage bone fastener 20′. Instrument 50 is shown engaged with side wall 28 to introduce rod 15 into the U-shaped channel 22. However, it is to be understood that instrument 50 is configured to engage either of side walls 26, 28, thereby permitting a surgeon to selectively introduce the rod 15 from either side of the bone fastener 20. Instrument 50 includes a first member 52 having a longitudinal axis 51. The first member 52 has a distal end 53 and a proximal end 55. The distal end 53 of first member 52 is configured to engage one of side walls 26, 28 for securing the instrument 50 to the bone fastener 20 such that the instrument 50 remains disposed outside of the passageway 11 of spinal rod 15, preferably in a manner that does not interfere with or block access to the U-shaped channel 22, the passageway 11 or the top opening 14. As can be seen in FIG. 1, the instrument 50 is disposed with respect to bone fastener 20 so as not to block top opening 14 of bone fastener 20 thereby advantageously avoiding interference with the introduction of spinal rod 15 in U-shaped channel 22. Preferably, distal end 53 may include jaws 54 for engaging one of the side wall 26, 28 of head 24.

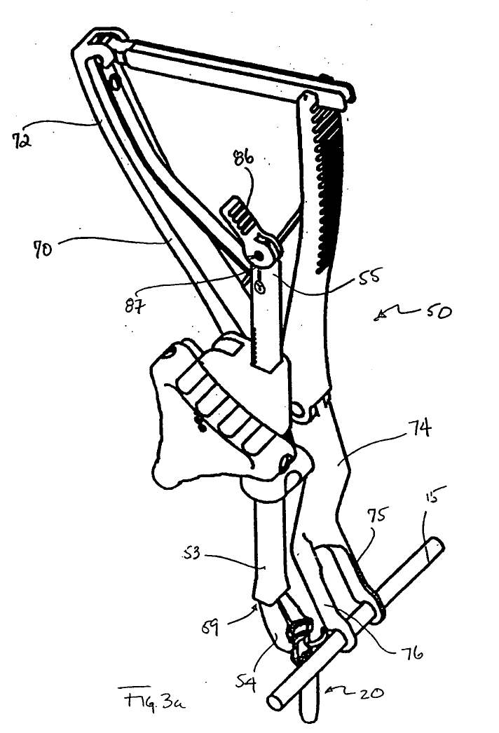

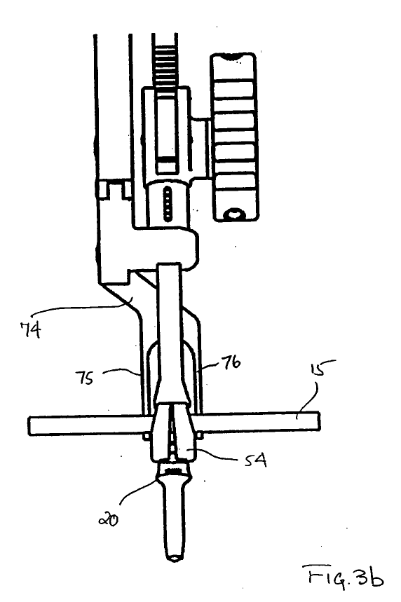

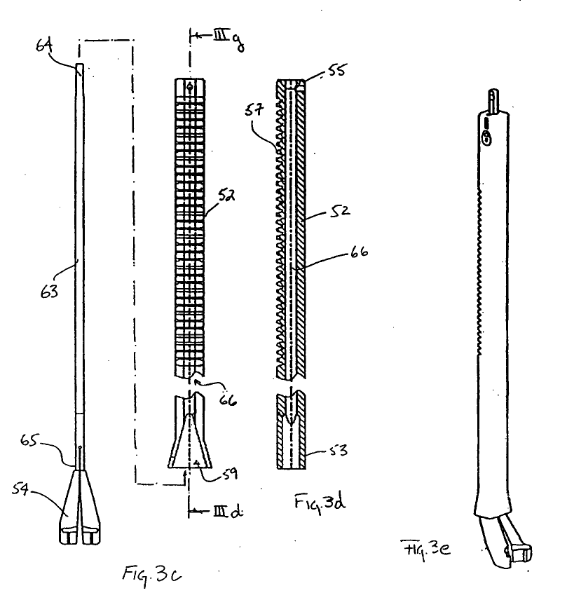

While FIGS. 1 and 3a-3i show jaws 54 separately disposed at the distal end 53 of first member 52, other configurations are within the scope of the present invention. For example, jaws 54 may be integrally formed at the distal end of first member 52. In a preferred embodiment, shown in FIGS. 3c-3e, first member 52 includes a through bore 66, and at its distal end 53 is a tapered chamber 59 that gets smaller in the direction of the proximal end 55. Received in the through bore 66 of second member 52 is elongated shaft 63, as shown in FIG. 3c. Elongated shaft 63 includes proximal end 64 and distal end 65. Disposed on the distal end 65 of elongated shaft 63 are jaws 54, wherein jaws 54 may be formed integrally or separately with respect to shaft 63. Shown in FIG. 3e is an assembled perspective view of shaft 63 housed in first member 52. Referring now to the assembled views of FIGS. 1 and 3a and the exploded view of FIG. 3i, proximal end 64 of shaft 63 is connected to a cam lock 86 to secure the shaft 63 in the bore of second member 52. Cam lock 86 having cam surface 87 forms a cammed engagement with the first member 52 at the proximal end 55. Upon rotation of cam lock 86 with respect to the proximal end 55, cam surface 87 slides along the proximal end 55 of first member 52, which translates shaft 63 with respect to the first member 52 in the direction towards the proximal end 55. The linear translation of shaft 63 causes the jaws 54 to engage the inner walls of tapering chamber 59 thereby causing the open biased jaws 54 to draw together to secure about the side wall 28 of bone fastener 20. Referring specifically to FIG. 3i, additionally disposed within chamber 59 and about shaft 63 may be a biasing element 69, such as, for example, a helical spring, for biasing the shaft 63 and jaws 54 downward relative to chamber 59 such that the jaws are biased into an open position when cam lock 86 is in the open position. Biasing element 69 is shown as a helical spring, however other biasing elements as is well known in the art are possible.

Shown in FIG. 3a is a perspective view of the engagement of instrument 50 with head 24 of bone fastener 20. A pair of jaws 54 are engaged with side wall 28 to secure the instrument 50 to bone fastener 20. Shown in FIG. 3b is a plan view of the engagement of the jaws 54 about the side wall 28. Shown in FIG. 3f is a top view of jaws 54, which may additionally include oppositely opposed flats or extensions 60, 62 for engaging recesses 42, 44 to further secure the hold of instrument 50 about the sidewall 28. The extensions 60, 62 may be configured or angled to engage the preferred recesses 42, 44, as previously described, on the end faces 34, 36. Thus, extensions 60, 62 may be accordingly dimensioned and configured for secure engagement with the end walls 35, 37 and seat 45. In addition, the dimensions and configurations of extensions 60, 62 may facilitate a mated engagement with end walls 35, 37 and seat 45. Preferably extensions 60, 62 are dimensioned with respect to L2 to form the secure engagement, or alternatively mated engagement, within recess 41, 42, 43, 44. In addition the jaws 54 may be dimensioned accordingly with respect to L1 so as to properly engage the length of side wall 26, 28. The secure engagement may form a close fit between the jaws 54 and the fastener 20 such that the fastener is restrained from rotation with respect to the jaws. Or where the bone fastener is anchored in bone, the secure engagement of the jaws 54 and extensions 60, 62 with the end walls 35, 37 and seat 45, prevent device 50 from slipping, moving or rotating with respect to the bone fastener. The jaws 54 and extensions 60, 62 are additionally dimensioned so as not to extend into the U-shaped channel 22 upon engagement of the jaws 50 about side walls 26, 28 thereby avoiding interference with installation of rod 15 in the U-shaped channel 22. The secure engagement of the jaws 54 and extensions 60, 62 with the recesses 42, 44 also locates instrument 50 clear of the passageway 11 of spinal rod 15 as was previously described and shown in FIG. 1. This assembled configuration facilitates rod installation in multiple bone fasteners 20 aligned along the spine where spinal rod 15 is interconnecting one bone fastener 20 to another bone fastener 20.

As seen in FIG. 3f, jaws 54 include an opening 61 defining an interior surface 56. Interior surface 56 may be configured to engage exterior side surface 40, and in a preferred embodiment, interior surface 56 may be substantially arcuate for secure engagement with the contour of exterior side surface 40 of side wall 28, which may be accordingly arcuate. The configuration of interior surface 56 may facilitate a mated engagement with the exterior side surface 40. The secure engagement of interior surface 56 with exterior side surface 40 further secures the bone fastener 20 with respect to the jaws 54, thereby further preventing rotation or movement of the bone fastener 20 with respect to the jaws 54. More specifically, the extensions 60, 62 have inclined surfaces angled accordingly with respect to α, as previously defined, so as to reach around the exterior surface 40 and mate with the seat 45. When the shaft 63 translates, which closes the jaws 54, the extensions 60, 62 engage the seat 45. As the extension 60, 62 engage the seat 45, the interior surface 56′ engages and contacts the exterior surface 40 to pinch the bone fastener 20 and hold the fastener by compression between the extensions 60, 62 and the interior surface 56. Shown in FIGS. 3g and 3h, respectively, are plan and side views of jaws 54 located at the distal end 65 of shaft 63.

Referring back to FIG. 1, instrument 50 further includes a second member 70 having a first end 72 and a second end 74. The second end 74 is configured for engaging and securely guiding the rod 15 through the passageway 11 and into the U-shaped channel 22. Shown in FIG. 1 and in greater detail in FIGS. 3a and 3b, second end 74 includes two arms 75, 76 having ends configured to engage and guide spinal rod 15 through passageway and into the U-shaped channel 22. Arms 75, 76 may be formed integrally with the second member 70 at the second end 74, or alternatively, the arms 75, 76 may be separably disposed at the second end 74, for example by a mechanical joint. Referring to FIG. 3b, arms 75, 76 are fixedly spaced from one another such that arms 75, 76 preferably equidistantly straddle head 24 when jaws 54 are secured about side wall 28, 26. Additionally, arms 75, 76 may be substantially angled with respect to at least a portion of the second member 70 or alternatively arms 75, 76 may be substantially angled with respect to one another.

Second member 70 is operably mounted with respect to first member 52 such that the second member 70 may pivot with respect to first member 52 so as to move the second end 74 and the rod 15 with respect to jaws 54. Therefore, with jaws 54 engaged with head 24, the second end 74 of second member 70 may be moved and positioned with respect to the head 24. As shown in FIG. 1, first end 72 of second member 70 may include pivotally engaged handle arms 71, 73. The first end 72 may be fixed or form one of the handle arms, for example handle arm 71, and first member 52 may be operably associated with the other handle arm 73. The configuration of arms 71, 73 permits second end 74 of the second member 70 to be pivotable with respect to the distal end 53 of the first member 52. The handles may be resiliently biased open by a spring element 77 located between the handle arms 71, 73. Second member 70 may further include, proximate the first end 72, a ratchet mechanism 90 for selectively positioning and fixing the second end 74 with respect to the first member 52.

Still referring to FIG. 1 and shown in greater detail in FIG. 3i in a preferred embodiment, the instrument 50 may additionally include a shaft or housing 80 having a through bore or channel 81, with the first member 52 received therein. In the preferred embodiment, second member 70 is pivotally mounted about the housing 80 such that second member 70 is pivotable with respect to the first member 52. Housing 80 may be formed from a single integral component, or housing 80 may be assembled from a plurality of interconnected modular components so as to form a continuous channel 81. Channel 81 may be dimensioned and configured so as to accordingly receive and house first member 52.

In the embodiment shown in FIG. 1, second member 70 is additionally operably associated with the first member 52 such that second member 70 can translate linearly along the longitudinal axis 51 between the proximal end 55 and the distal end 53 of first member 52. Referring to FIGS. 1, 3c-3e and 3i linearly disposed along at least a portion of first member 52 between the first end 55 and the second end 53 may be a rack or external gear teeth 57. Shown in FIG. 3i, is a pinion 82 having a knob 84, which may be mounted about the housing 80. Pinion 82 is operatively engaged with gear teeth 57 of first member 52 by a pawl or switch 85 disposed therebetween such that upon rotation of knob 84, pinion 80 engages gear teeth 57, via switch 85, so as to linearly translate housing 80 and second member 70 with respect to first member 52 along the longitudinal axis 51. Switch 85 may be configured to toggle between a first position such that the second member 70 may translate only in a first direction relative to the first member 52 and a second position where the second member 70 may translate only in second direction opposite of the first direction. Switch 85 may have a third position where the second member 70 is locked relative to the first member and a fourth position where the second member is translatable in either direction along the first member 52. While FIGS. 1, 3a and 3i show instrument 50 having a separate mounted housing 80 with a rack and pinion mechanism 57, 82, other mechanisms known in the art are possible for providing both pivotable and linear engagement between the first member 52 and the second member 70.

Instrument 50 as shown in FIG. 1 may operate as follows. Bone fastener 20 is affixed to bone, preferably the pedicle of a vertebral bone. Instrument 50 attaches to bone fastener 20, more specifically, to one of the side walls 26, 28 in a manner as previously described. With the jaws 54 engaged about one of the side walls, for example, side wall 28, cam lock 86 is rotated thereby translating shaft 63 and jaws 54 in an upward direction along longitudinal axis 51 of first member 52. Resiliently open-biased jaws 54 engage the interior surface of conically tapering chamber 59 such that the jaws 54 close about the side wall 28 thus securing instrument 50 to bone fastener 20. The arms 74, 75 located at the second end of second member 70 engage the spinal rod 15, and the articulating handles forming first end 72 of second member 70 are squeezed together so as to guide rod 15 in a direction substantially lateral and normal to the longitudinal axis 23 so as to position the spinal rod 15 directly above the U-shaped channel 22. The inclusion of ratchet mechanism 90 facilitates selective positioning of rod 15 relative to head 24 of bone fastener 20. With the rod located directly above and parallel to the elongated U-shaped channel 22, knob 84 may be rotated so as to engage the pinion 82 with gear teeth 57 disposed about first member 52, via switch 85, such that second member 70 is translated along longitudinal axis 51 in the direction of head 24 so as to introduce rod 15 into the U-shaped channel 22 substantially along the longitudinal axis 25 of bone fastener 20.

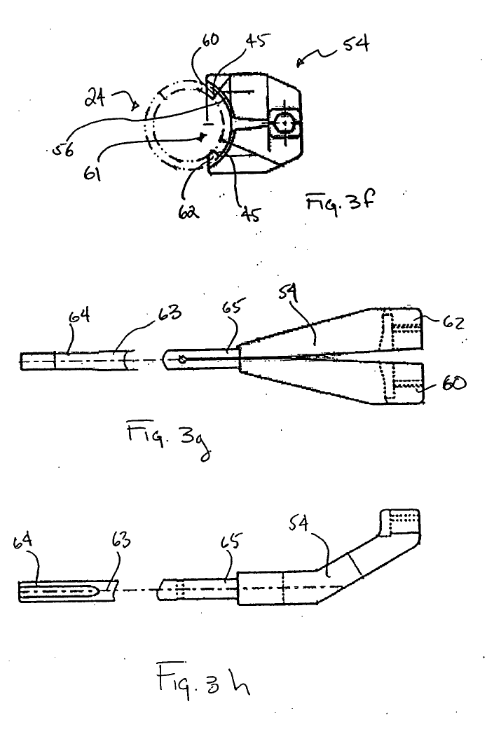

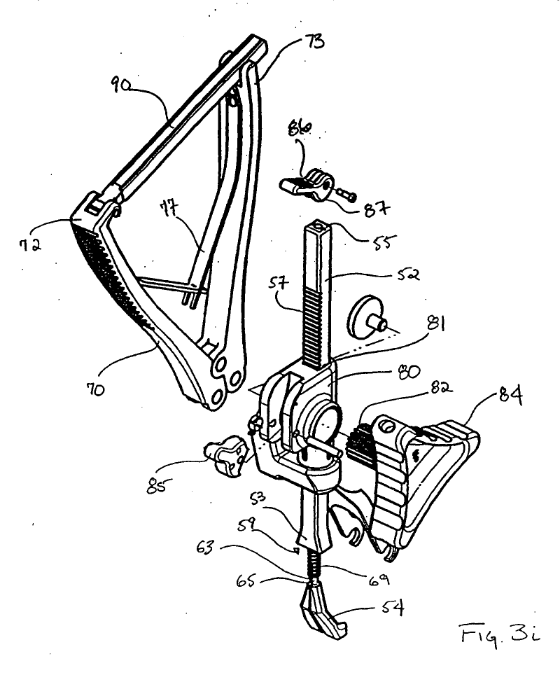

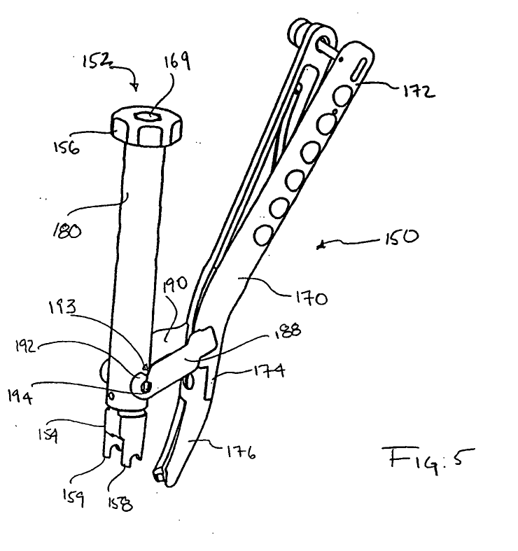

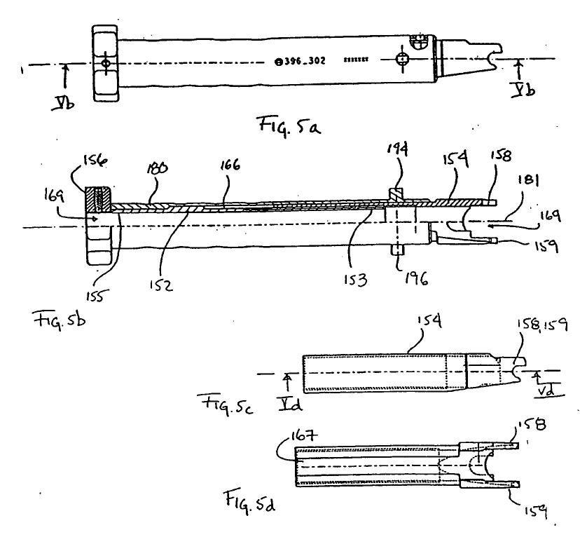

Referring now to FIG. 5 is a second illustrative embodiment of a rod introduction instrument shown as instrument 150. Instrument 150 includes a first member 152 for engaging and guiding spinal rod 15 through the passageway 11 and into the U-shaped channel 22, a second member 170 for securing instrument 150 to bone fastener 20, and a sleeve or shaft 180. Shown in FIG. 5 and FIGS. 5a-5d, are first member 152 and shaft 180. Partial cross-sectional view FIG. 5b shows shaft 180 having a central through bore 166 and longitudinal axis 181. First member 152 includes proximal end 155 and distal end 153 of first member 152 is received in through bore 166 of shaft 180. Distal end 153 is configured so as to engage spinal rod 15. Distal end 153 includes rod engaging tip 154. In a preferred embodiment, at least a portion of distal end 153 is threaded for threaded engagement with rod engaging tip 154. Rod engaging tip 154 is configured so as not to rotate with respect to shaft 180. Shown in greater detail in FIGS. 5c-5d, rod engaging tip 154 defines a central threaded bore 167 for the threaded engagement with distal end 153 of first member 152. At the distal end 157 of tip 154 are parallel rod engaging arches 158 and 159. Shown in FIG. 5d, arches 158, 159 are spaced from one another relatively equidistantly from central axis 181. Arches 158, 159 are shown in FIG. 5c as being integral with the remainder of rod engaging tip 154, but other configurations are possible. Referring back to FIG. 5b, proximal end 155 of first member 152 has a knob 156. With assembled first member 152 and rod engaging tip 154 received within shaft 180, rotation of knob 156 causes rod engaging tip 154 to move along the longitudinal axis 181 with respect to both the first member 152 and shaft 180 in the direction of the distal end 153. Alternatively, rod engaging tip 154, first member 152 and shaft 180 may be configured such that the rod engaging tip 154 linearly translates with respect to shaft 180 by a rack and pinion mechanism similar to the one of instrument 50 (not shown). The rack and pinion mechanism may include a switch configured to toggle between a first and second position to limit the direction of translation of rod engaging tip 154 relative to the shaft 180, a third position to lock rod engaging tip 154 relative to shaft 180, and fourth position to permit translation of the rod engaging tip 154 in either direction relative to the shaft 180. In addition, first member 152 and knob 156 may form a continuous channel 169 extending from the proximal end 155 to the distal end 153. Surgical instruments, for example, a tightening tool configured for securing the locking cap 700 may be inserted through the channel 169 to secure the locking cap 700 and the instrument 150 may remain in place without interfering with the installation of the locking cap 700.

Referring back to FIG. 5, instrument 150 includes second member 170 having a first end 172 and a second end 174, with the second end 174 configured for securing instrument 150 to bone fastener 20. Shown in FIGS. 5e and 5f are a top and plan views of second member 170. The second end 174 includes jaws 176. Jaws 176 are configured similar to the jaws 54 of instrument 50, as previously described, for engaging side wall 26, 28 of bone fastener 20. Specifically, jaws 176 include extensions 160, 162, which are configured for secure engagement with the recesses 41, 42, 43, 44 of side walls 26, 28 of bone fastener 20. Jaws 176 also includes opening 161 and defines interior surface 151 for secure engagement with exterior side surfaces 38, 40. The jaws 176 differ from jaws 54 of instrument 50 only with respect to the manner in which the jaws are drawn together to secure about side wall 26, 28 of bone fastener 20. Jaws 176 are pivotally drawn together whereas jaws 54 of instrument 50 are resiliently biased open and closed by interference with the conically tapering chamber 59. In a preferred embodiment, first end 172 of second member 170 is formed by a pair of pivotally connected arms 178, 179. The arms 178, 179 are preferably biased open by a resilient element, for example a spring 182. Also included in the preferred embodiment is a threaded rod 184 having an adjustment knob 186, disposed proximate the first end 172, for drawing together and holding in a fixed relationship, arms 178, 179 so as to secure one of the side walls 26, 28 in the jaws 176. The knob 186 ceases to be threadably advanceable along threaded rod 184 once arms 178, 179 have drawn jaws 176 to a secure closed position. As with instrument 50, the engagement of jaws 176 with one of the side walls 26, 28 fixes instrument 150 with respect to bone fastener 20 such that instrument 150 is substantially outside of the passageway 11 of spinal rod 15 so as not to interfere with the introduction of the rod 15 into the U-shaped channel 22.

Referring again to FIG. 5, second member 170 is pivotally engaged with shaft 180. In a preferred embodiment, second member 170 includes two parallel brackets 188, 190 attached to second member 170 between the first end 172 and the second end 174. Shown in plan view in FIG. 5e, brackets 188, 190 are spaced so as to accommodate shaft 180 therebetween, and shown in FIG. 5f, brackets 188, 190 are configured for the pivotable movement of shaft 180 relative to second member 170. Specifically, brackets 188, 190 may include a hook 192 having a slotted opening 193 for engagement with diametrically opposed pin members 194, 196 located on shaft 180, as most clearly shown in FIG. 5b. Referring again to FIG. 5f, slotted opening 193 of hook 192 may be located on the top side of brackets 188, 190 such that the shaft 180 may be introduced into engagement with brackets 180, 190 from the top. Alternatively, slotted opening 193 may be located on the bottom side of brackets 180, 190 (not shown) for introduction of shaft 180 from the bottom.

The instrument 150, shown in FIG. 5 may operate as follows. Bone fastener 20, for example, is anchored to, preferably, the pedicle of a vertebral bone. The instrument 150 attaches to one of the side walls 26, 28 of bone fastener 20. Instrument 150 is understood to similarly engage the head 24 of bone fastener 20′. Jaws 176 are brought into secure engagement with one of the side walls of bone fastener 20, for example, side wall 28 such that instrument 150 is clear of passageway 11 of spinal rod 15, preferably in a manner that does not interfere with or block access to the U-shaped channel 22, the passageway 11 or the top opening 14. Knob 186 is rotated, thereby selectively adjusting the articulation of arms 178, 179, via threaded rod 184, to a fixed distance of separation, so as to grip jaws 176 about side wall 28. With shaft 180 pivotally engaged with brackets 188, 190, the assembled shaft 180 and first member 152 may be pivoted with respect to the second member 170 so as to position the rod engaging tip rod 15 into engagement with the spinal rod 15 in a manner that will target and introduce spinal rod 15 into the elongated U-shaped channel 22. With the rod 15 in position, knob 156 may be rotated thereby causing linear translation of rod engaging tip 154 with respect to the first member 152 and shaft 180 in the direction of the head 24 so as to introduce rod 15 into U-shaped channel 22 in a direction substantially coaxial with the first member 152.

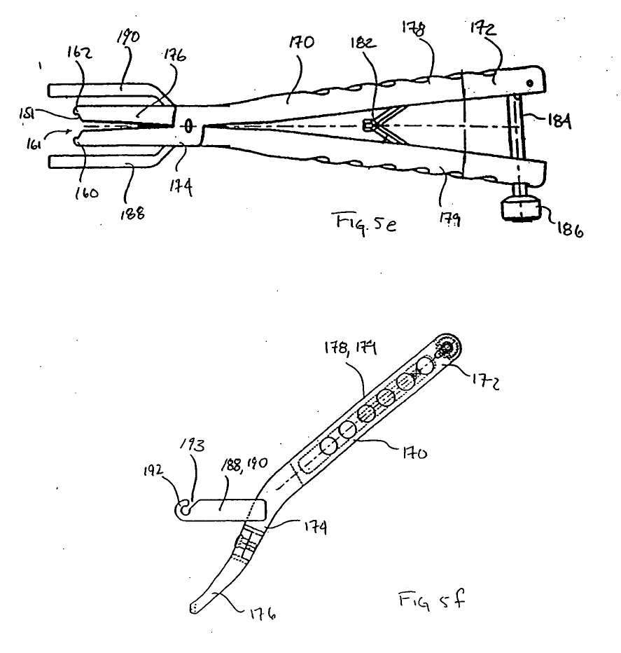

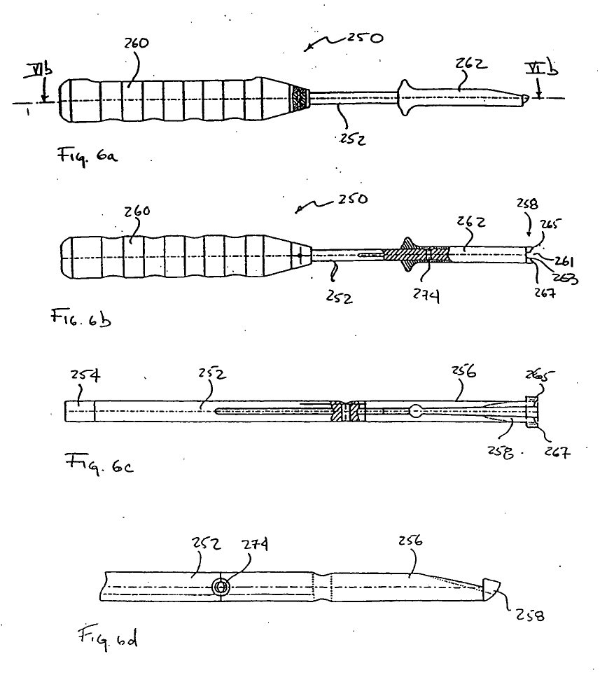

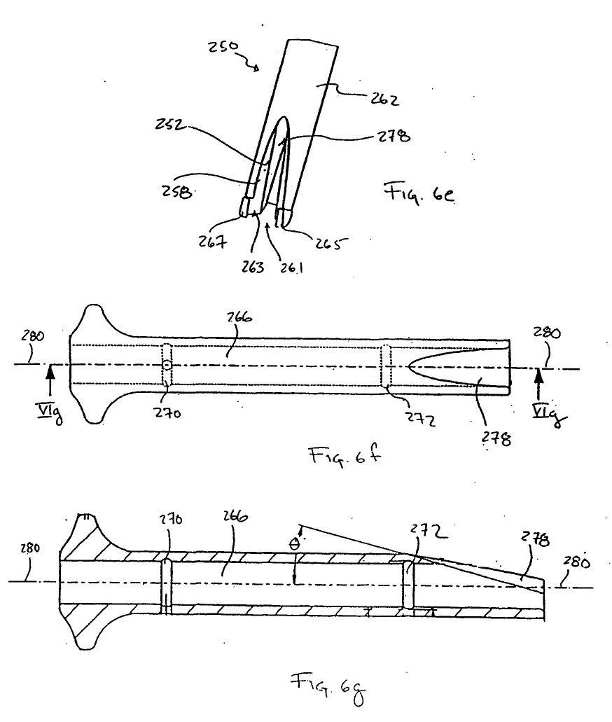

Shown in FIGS. 6a-6b is another instrument 250 for the handling and manipulation of bone fasteners similarly configured to bone fastener 20. Instrument 250 comprises an elongated member 252, a handle 260 and a shaft 262. Shown in greater detail in FIG. 6c is elongated member 252 having a proximal end 254 and a distal end 256 configured for engaging and securely holding the head of a bone fastener 20. A handle 260 is disposed about or attached to the proximal end 254. Disposed about or attached to the distal end 256 are jaws 258. In FIG. 6c, jaws 258 are shown as being formed integral with the distal end 256, but other configurations are possible. The jaws 258 shown in FIGS. 6c-6e, are configured for mating engagement with side wall 26, 28 of head 24 of bone fastener 20 in a manner substantially similar as previously described. Jaws 258 may include extensions 265, 267 configured for secure engagement with recesses 41, 42, 43, 44 of side wall 26, 28 of bone fastener 20. The jaws also define opening 261 and may further define interior surface 263 for secure engagement with exterior side surfaces 38, 40 of side walls 26, 28. Jaws 258 may be configured so as to prevent rotation of bone fastener 20 with respect to instrument 250 upon engagement of the jaws 258 with one of the side walls 26, 28.

Disposed about elongated member 252 between the proximal end 254 and the distal end 256 is shaft 262, shown in FIGS. 6f-6g, having a central channel or through bore 266, the shaft 262 is permitted to slide along elongated member 252. Through bore 266 is shown as substantially cylindrical but other configurations are possible, for example, a substantially rectangular volume. Shaft 262 has a first proximal position in which the jaws 258 are resiliently biased open, and shaft 262 has a second distal position, proximate the distal end 256, in which the jaws 258 are drawn closed. As shaft 262 slides from its first proximal position to the second distal position, the arms forming jaws 258 engage the interior wall of shaft 262 drawing the jaws 258 closer together into the closed position. Shown in cross-section in FIG. 6f, shaft 262 includes central through bore 266, which is dimensioned so as to receive elongated member 252 and distal end 256. The central through bore 266 is dimensioned so as to draw jaws 258 closed when shaft 262 is in the second distal position, an exemplary diameter of central through bore 266 may be approximately 8 mm. Central through bore 266 may additionally include grooves 270, 272 for engagement with a pin member 274, as seen in FIGS. 6b, 6d, inserted normal to the longitudinal axis of elongated member 252. Grooves 270 and 272 are spaced relative to one another so as to locate the first and second positions of shaft 262 upon respective engagement with pin member 274. Referring again to FIGS. 6e-6g, shaft 262 may be configured so as to include an opening 278 at the distal end 276 of shaft 262. Opening 278 is configured so as to permit the jaws 258 to securely engage side walls 26, 28 of bone fastener 20 without substantial interference from shaft 262 as the jaws 258 are operably drawn from the open to close position when shaft 262 is moved from the first proximal position to the second distal position. The opening 278 may have a substantially arched-shaped opening, as seen in the plan view of FIG. 6f, and a portion of the opening 278 may be at an angle θ with respect to the longitudinal axis 280 of the shaft 262 as seen in the cross-sectional side view of FIG. 6g. Angle θ may preferably be about 15° with respect to the longitudinal axis 280, however other angles for angle θ are possible.

In operation, a surgeon can grasp instrument 250 by the handle 260 and the jaws 258 are brought into engagement with one of the side walls 26, 28 of the head 24 of, for example, bone fastener 20′. Bone fastener 20 may be similarly engaged. The jaws 258 in an open configuration, approach either of side walls 26, 28 in a direction substantially normal to the longitudinal axis 25 of the bone fastener 20′. The substantially normal approach of the jaws 258 ensures alignment and proper engagement of the jaws 258 with either recesses 41, 43 of side wall 26 or recesses 42, 44 of side wall 28. The opposed extensions 265, 267 of jaws 258 may be spaced relative to one another such that angled approaches of jaws 258 toward side wall 26, 28 may result in interference with the head 24 so as to prevent proper engagement of jaws 258 with, for example, recesses 42, 44 of side wall 28. With the jaws 258 properly aligned with the recesses 42, 44, the shaft 262 is slid along the elongated member 252 from the first proximal position to the second distal position thereby causing an interference fit between the through bore 266 and the distal end 256 of the elongated member 252 so as to draw jaws 258 into the closed position about the side wall 28 with the extensions 265, 267 in secured engagement with recesses 42, 44. The mating engagement between the jaws 258 and the side wall 28 secures the bone fastener 20′ in the instrument 250 for manipulation and placement during a surgical procedure.

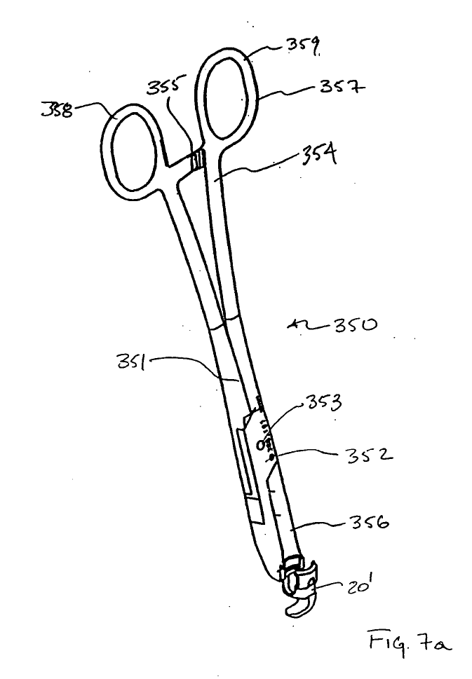

Now referring to FIG. 7a, another illustrative embodiment, instrument 350, also for use in the handling and manipulation of bone fasteners configured as, for example, bone fastener 20′. Instrument 350 comprises an elongated member 351 having a distal end 352 configured for engaging and securely holding a bone fastener 20 and a proximal end 354 including a handle 357 for operating instrument 350. Distal end 352 includes jaws 356, and shown in FIG. 7b, jaws 356 are configured substantially similarly to jaws 258 of instrument 250 and the other instruments previously described, for secure engagement with and about one of the side walls 26, 28 of bone fastener 20 so as to remain clear of passageway 11 of spinal rod 15. Jaws 356 includes extensions 360, 362 with an opening 361 further defining an interior surface 368. The main difference between jaws 356 and jaws 258 is the manner is which the jaws are moved to the closed position. Referring again to FIG. 7a, jaws 356 are operably pivotally engaged whereas, previously described, jaws 258 are resiliently biased open and closed by the interference with the interior of shaft 262. To open and close jaws 356, instrument 350 includes a handle 357 located at the proximal end 354. In a preferred embodiment, the handle 357 is formed by forceps handles 358, 359 which are pivotally connected about pin 353 to articulate jaws 356 between the open and closed position. Each leg of jaw 356 may be formed integrally with one of the handles 358, 359, in which the jaws 356 and handles 358, 359 are operably connected by a single pivoting connection. In a preferred embodiment, handles 358, 359 may include a ratchet mechanism 355 for selectively spacing apart jaws 356 at a fixed distance.

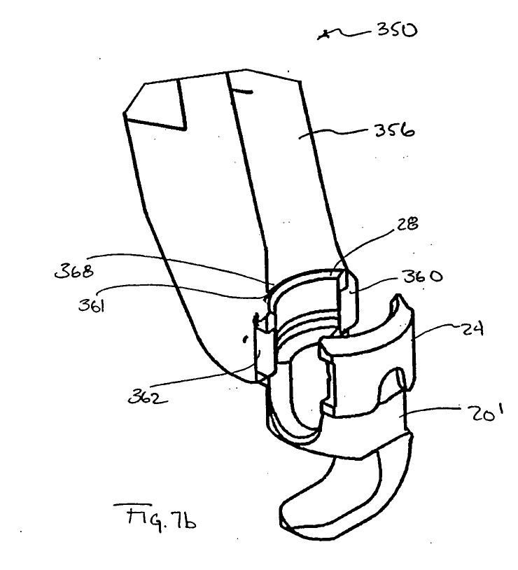

Now referring to FIG. 8a, shown is another instrument 450 for handling and manipulating bone fasteners configured similarly to bone fastener 20′. Instrument 450 comprises an elongate member 451 having a distal end 452 configured for securely engaging the head 24 of bone fastener 20′, and a proximal end 454 for operating instrument 450. Distal end 452 has jaws 456, which are configured for engaging head 24 such that jaws 456 straddle head 24 about U-shaped channel 22, as shown in FIG. 8b, in a manner that aligns instrument 450 substantially over the top of the passageway 11 of the spinal rod 15. Referring to FIG. 8b, the jaws 456 are formed by parallel legs 458, 460 defining an interior chamber or space 462 which is configured to accommodate the head 24, the head being substantially circular cylindrical. In a preferred embodiment, each of the parallel legs 458, 460 may include extensions 464, 466 for engaging recesses 46, 48 defined by the external side surfaces 38 and 40, as shown in FIGS. 2a, 2c. Jaws 456 are pivotally operable, and in a preferred embodiment, the handles of instrument 450 at the proximal end 454 are formed as forceps handles 468, 470. The handles 468, 470 are pivotally operable, by articulating jaws 456 between the open and closed position. Each leg 458, 460 of jaws 456 may be formed integrally with one of the handles 468, 470, such that jaws 456 and handles 468, 470 are operably connected by a single pivoting connection 453. In a preferred embodiment, handles 468, 470 may include a ratchet mechanism 472 for selectively spacing apart jaws 456 at a fixed distance with respect to one another.

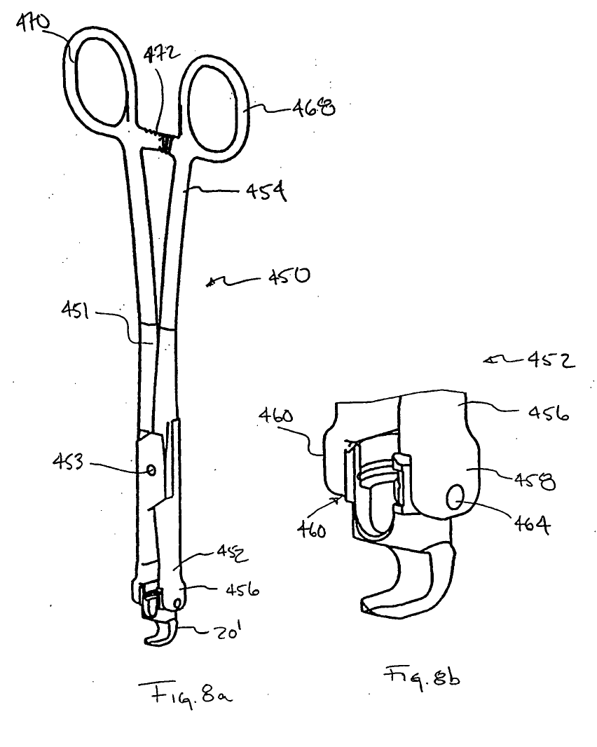

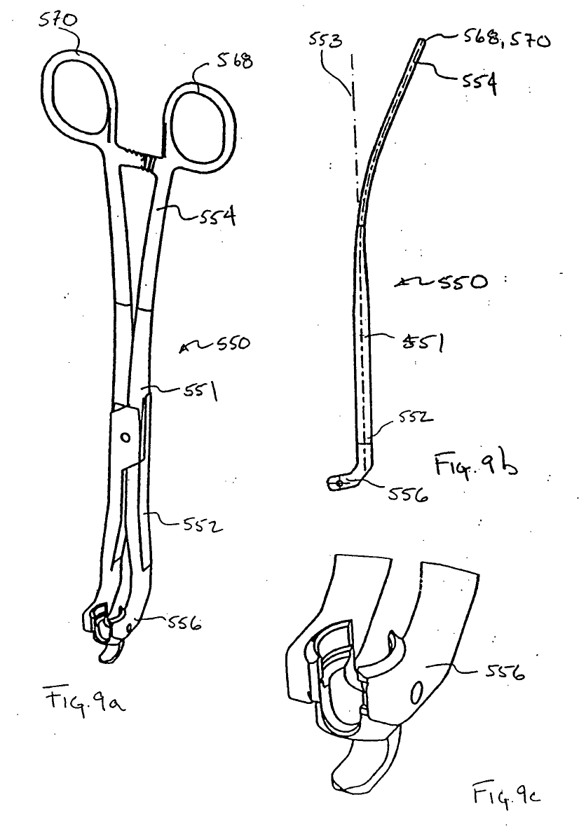

Now referring to FIGS. 9a-9b, shown is instrument 550, an alternative embodiment of the instrument 450. Elongated member 551 defines a longitudinal axis 553, elongated member 551 includes a distal end 552 configured for engaging head 24 of bone fastener 20 and a proximal end 554. Distal end 552 has jaws 556 which are substantially similarly configured to jaws 456 of instrument 450. Jaws 556, shown in FIG. 9c, similarly engage head 24 of a bone fastener 20 such that the jaws straddle about U-shaped channel 22, thereby aligning instrument 550 over the top of the passageway 11 of spinal rod 15. In a preferred embodiment, the proximal end 554 is formed by forceps handles 568, 570. A portion of forceps handles 568, 570 may be bent or angled such that proximal end 554 lies outside of a plane that includes longitudinal axis 553, thus proximal end 554 is disposed angularly with respect to the remainder of instrument 550, or alternatively, proximal end 554 may be arcuately disposed with respect to the remainder of instrument 550.

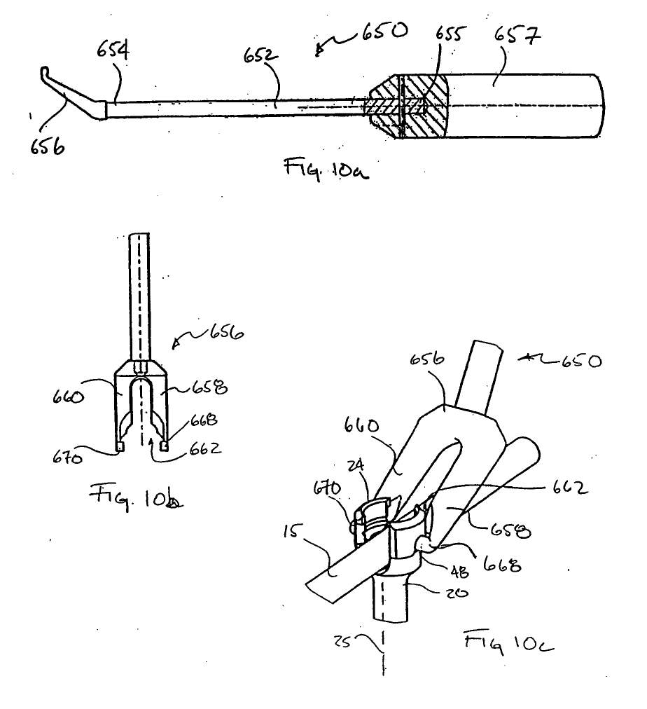

Now referring to FIG. 10a, shown is another embodiment, instrument 650 for introducing a spinal rod 15 into the elongated U-shaped channel 22 of bone fastener 20. Instrument 650 comprises a shaft 652 having a distal end 654 for engaging both the rod 15 and the head 24 of bone fastener 20, the shaft 652 further having a proximal end 655. Instrument 650 further comprises handle 657 disposed about, integral with or attached to the proximal end 655 of shaft 652 for manipulating instrument 650. Disposed about, integral with or attached to the distal end 654 is prong 656 for simultaneous engagement with both rod 15 and head 24. Shown in FIG. 10b, prong 656 includes substantially rigid parallel legs 658, 660 defining an interior surface 662 configured for mating engagement with rod 15. In a preferred embodiment, disposed about the distal ends of legs 658, 660 may include opposed extensions 668, 670 configured for engaging cavities 46, 48 of exterior side surfaces 38, 40, or more specifically with shoulders 47, 49, shown in FIG. 2c. Opposed extensions 668, 670 may be substantially circular-cylindrical, but other configurations are possible, for example, extensions 668, 670 may be configured such that only a portion of the extension 668, 670 is arcuate or rounded for engagement with shoulders 47, 49. In addition extensions 668, 670 may be dimensioned accordingly based upon the depth D₁ of recess 46, 48 so as to ensure proper engagement with the shoulders 47, 49.

In operation of instrument 650, shown in FIG. 10c, the spinal rod 15 is initially placed atop the head 24 between legs 26, 28 at the opening of U-shaped channel 622. Prong 656 is brought over top of rod 15 at an approach offset from axis 25 of bone fastener 20 and initially located beneath the head 24 of bone fastener 20. Extensions 668, 670 are aligned with recesses 46, 48 beneath head 24 such that the handle 657 is substantially aligned with the passageway of spinal rod 15. The instrument 650 is pulled in the upward direction along longitudinal axis 25 of bone fastener 20 such that the extensions 668, 670 engage shoulders 47, 49. Due to the rigidity of the legs 658, 660, the extensions 668, 670 are preferably aligned with the recesses 46, 48 prior to engagement with shoulders 47, 49 in order to avoid interference with portions of the head 24 of bone fastener 20 that are wider in dimension than the spacing between legs 658, 660. With the interior surface 662 engaged with rod 15, handle 655 is actuated so as to pivot instrument 650 about recesses 46, 48. With the engagement of extensions 668, 670 with shoulders 47, 49 providing leverage, the pivoting action of instrument 650 creates a cammed action between interior surface 662 and rod 15, such that rod 15 is driven or pivoted in a downward direction along longitudinal axis 25 into the U-shaped channel 22. Instrument 650, with its simple construction and absence of components which move relative to one another, relies on pivoting leverage to introduce spinal rod 15 into the U-shaped channel 22 of bone fastener 20.

The above description of instruments involved in the handling and manipulation of bone fasteners were described as operating independent of one another, however it should be understood that the instruments may be collectively used in the course of single surgical procedure and may be manufactured and sold separately or together in the form of a surgical kit.

Further it should be understood that variations and modifications within the spirit and scope of the invention may occur to those skilled in the art to which the invention pertains. Accordingly, all modifications attainable by one versed in the art from the disclosure set forth herein are to be included as further embodiments of the present invention. The scope of the present invention is accordingly defined as set forth in the appended claims.

What is claimed:

1. A bone fastener for receiving a spinal rod, the bone fastener comprising:
a head having a top end, a bottom end, and first and second side walls;
the first and second side walls each having a first end face and a second end face, the second end face opposite the first end face, the first and second side walls defining an opening in the top end and a U-shaped channel through the head extending from the first end face to the second end face, the U-shaped channel having a longitudinal axis defining a passageway for receiving the spinal rod;
a bone engaging element for anchoring to bone, the bone engaging element having a central axis, the bone engaging element extending from the bottom end of the head; and
wherein, each of the first and second end faces of the first and second side walls includes a recess configured for secure engagement with an instrument for handling and manipulating the bone fastener and spinal rod such that the instrument does not interfere with the opening at the top end.

2. The bone fastener of claim 1, wherein the recess is in communication with the U-shaped channel.

3. The bone fastener of claim 1, wherein each of the recesses of the first and second end faces includes a seat for secure engagement with the instrument.

4. The bone fastener of claim 3, wherein each of the seats defines an angle with respect to the longitudinal axis of the U-shaped channel.

5. The bone fastener of claim 4, wherein the angle is about 15° to about 90°.

6. The bone fastener of claim 4, wherein the angle is about 45° to about 75°.

7. The bone fastener of claim 4, wherein the angle is about 55° to about 60°.

8. The bone fastener of claim 1, wherein each of the recesses of the first and second end faces further defines a first end wall and a second end wall, the seat located therebetween, wherein the first and second end walls are dimensioned and configured for secure engagement with the instrument to prevent the fastener from pivoting with respect to the instrument.

9. The bone fastener of claim 1, wherein the first and second side walls have an exterior side surface that is substantially arcuate.

10. The bone fastener of claim 1, wherein each of the first and second side walls define an interior side surface and an exterior side surface, the interior side surface further defining the U-shaped channel and the exterior side surface located opposite the internal surface.

11. The bone fastener of claim 9, wherein the exterior side surfaces of the first and second side walls includes a cavity configured for engaging an instrument for handling the bone fastener and spinal rod.

12. The bone fastener of claim 11, wherein the cavity varies in depth from the top surface to the bottom surface.

13. The bone fastener of claim 11, wherein the cavity defines a shoulder for engaging the instrument applying a force in a direction along the central axis.

14. The bone fastener of claim 9, wherein the interior surface is threaded for engaging an externally threaded cap to retain the spinal rod in the U-shaped channel.

15. The bone fastener of claim 1, wherein the bone engaging element is integral with the head of the bone fastener.

16. The bone fastener of claim 1, wherein the bone engaging element is a threaded shank.

17. The bone fastener of claim 1, wherein the bone engaging element is a hook.

18. The bone fastener of claim 1, wherein the bone engaging element is separably engaged with the bottom surface of the head, the bone engaging element having a proximal end and a distal end, the distal end being configured for anchoring to the bone.

19. The bone fastener of claim 18, wherein the head further includes an internal cavity configured for securely receiving the proximal end of the bone engaging element such that the bone engaging element lies at a surgical selected angle relative to the head.

20. A system for introducing an elongated spinal rod, the system comprising:
a bone fastener having;
a head, the head including a top end, a bottom end, a pair of side walls connected at the bottom end defining an elongated U-shaped channel having a first longitudinal axis and a top opening located at the top end, the top opening and the channel further defining a passageway configured for receiving the spinal rod, each side wall further defining a first end face and a second end face, the second end face opposite the first end face; and
a bone engaging element extending from the bottom surface of the head for anchoring the bone fastener to the bone;
the system further comprising a tool for introducing the rod into the U-shaped channel, the tool including:
a first member having a distal end and a proximal end, the distal end configured for guiding the rod through the passageway to introduce the rod into the U-shaped channel through the top opening,
a second member defining a second longitudinal axis substantially perpendicular to the first longitudinal axis, the second member having first and second ends, the second end having a pair of jaws for securing the tool to either one, but only one, of the side walls of the head of the bone fastener such that second member is disposed substantially outside of the passageway of the spinal rod; and
wherein the first member is operably associated with the second member such that the first member is pivotable with respect to the second member and linearly translatable in a direction parallel to the second longitudinal axis between the first and second ends of the second member along the second longitudinal axis for introducing the spinal rod through the top opening into the passageway.

21. The system of claim 20, wherein the first and second end faces each define a recess, the jaws include a first extension and a second extension, the second extension located opposite the first extension, wherein further the first extension is configured to engage the recess of the end face and the second extension is configured to engage the recess of the second end face to fix the instrument with respect to the fastener.

22. The system of claim 21, wherein each of the recesses of first and second end faces further define a seat and two opposed end walls dimensioned and configured for secure engagement with one of the first and second extensions, wherein each of the seats form an angle with respect to the longitudinal axis of the U-shaped channel.

23. The system of claim 22, wherein the angle is about 15° to about 90°.

24. The system of claim 22, wherein the angle is about 45° to about 75°.

25. The system of claim 22, wherein the angle is about 55° to about 60°.

26. The system of claim 20, wherein the side walls further define an exterior side surface and the jaws define an interior surface, the interior surface being configured for secure engagement with the exterior side surface so as to prevent rotation of the bone fastener with respect to the instrument.

27. The system of claim 26, wherein the exterior side surfaces are substantially arcuate, and wherein further the interior surface of the jaws is substantially arcuate being configured for secure engagement with the exterior side surface.

28. The system of claim 20, wherein the bone engaging element is integral with the head.

29. The system of claim 20, wherein the head of the bone fastener further comprises an internal cavity and the bone engaging element further comprises a proximal end and a distal end, the distal end being configured for anchoring to bone, wherein the cavity is configured for securely receiving the proximal end for fixing the bone engaging element at a surgeon selected angle with respect to the head.

30. An instrument for introducing an elongated spinal rod into a U-shaped channel of a bone fastener having a top opening, the bone fastener having a pair of side walls defining the U-shaped channel and the top opening to the U-shaped channel, the instrument comprising:
a first member having a longitudinal axis, a proximal end and a distal end configured for engaging one of the side walls to secure the instrument to the fastener such that the instrument is disposed outside of the passageway of the spinal rod;
a second member having a first end and a second end, the second end configured for guiding the spinal rod into the U-shaped channel;
wherein the second member is operably associated with the first member such that the second end of the second member is pivotable with respect to the distal end of the first member and the second member is linearly translatable along the longitudinal axis with respect to the first member for introducing the spinal rod into the U-shaped channel.

31. The instrument of claim 30, wherein each of the side walls of the bone fastener include a first and second end face, each first and second end face defining a recess;
wherein further the distal end of the first member includes a pair of jaws, the jaws having a first extension and a second extension, the second extension located opposite the first extension, wherein further the first extension is configured to engage the recess of the first end face and the second extension is configured to engage the recess of the second end face.

32. The instrument of claim 31, wherein each of the recesses defines opposing end walls and a seat therebetween, the seats forming an angle with respect to a longitudinal axis defined by the U-shaped channel;
wherein further the first and second extension are dimensioned and configured so as to form a secure engagement with the opposing end walls and the seat to prevent rotation of the fastener with respect to the jaws.

33. The system of claim 32, wherein the angle is about 15° to about 90°.

34. The system of claim 32, wherein the angle is about 45° to about 75°.

35. The system of claim 32, wherein the angle is about 55° to about 60°.

36. The instrument of claim 31, wherein the side walls of the bone fastener include an exterior side surface, and the jaws define an interior surface configured for mating engagement with the exterior side surface to prevent rotation of the bone fastener with respect to the jaws.

37. The instrument of claim 30, wherein the side walls and the exterior side surface are substantially arcuate, and wherein further the interior surface of the jaws is substantially arcuate for secure engagement with the exterior side surface.

38. The instrument of claim 30, further comprising a housing having a through channel, the first member received in the through channel, the housing linearly translatable along the longitudinal axis of the first member, and the second member pivotally mounted to the housing such that the second end of the second member is pivotable and linearly translatable with respect to the distal end of the first member.

39. The instrument of claim 30, wherein the first member has an external surface at least a portion of which has gear teeth,
the instrument further comprising an assembly comprising a pinion, a knob and a switch, the pinion and switch threadably engaging the gear teeth of the first member and operably associated with the second member for translating the second member with respect to the first member upon rotation of the knob.

40. The instrument of claim 39, wherein the switch has a first position in which the second member is limited to translation relative to the first member in a first direction, the switch having a second position in which the second member is limited to translation in a second direction opposite the first direction.

41. The instrument of claim 40, wherein the switch has a third position in which the second member is fixed relative to the first member and a fourth position in which the switch permits translation of the second member in either direction along the longitudinal axis of the first member.

42. The instrument of claim 30, wherein the first member comprises a central through channel defining a chamber at the distal end of the first member, the first member further comprising an elongated member having a proximal end and a distal end received in the central channel of the first member, wherein the jaws depend from the distal end of the elongated member and at least a portion of the jaws are located in the chamber.

43. The instrument of claim 42, wherein the instrument further comprises a cam lock associated with the proximal end of the elongated member for securing the elongated member in the central bore of the first member, and
wherein rotation of the cam lock translates the elongated member linearly with respect to the first member such that the jaws engage the chamber to close the jaws.

44. The instrument of claim 43, wherein the second member includes a pair of arms associated with the second end and configured for guiding the rod into the U-shaped channel.

45. The instrument of claim 44, wherein the arms are angled with respect to the second member.

46. The instrument of claim 44, wherein the arms are spaced relative to one another such that the head of the bone fastener may be accommodated between the arms.

47. The instrument of claim 30, further comprising a pair of pivotally connected handles, the second member fixed with respect to one handle, the first member operably associated with the other handle such that the second end of the second member is pivotable with respect to the distal end of the first member.

48. The instrument of claim 47, wherein the second member further comprises a ratchet mechanism associated with the pair of articulating handles, the ratchet mechanism configured to selectively pivotally position the second end of the second member in a fixed relationship with respect to the distal end of the first member.

49. The instrument of claim 47, wherein the first end of the second member forms the handle that is fixed with respect to the second member.

50. An instrument for introducing an elongated spinal rod into a U-shaped channel of a bone fastener having a top opening, the bone fastener having a pair of side walls defining the U-shaped channel and the top opening to the U-shaped channel, the instrument comprising:
a first member including a shaft, at least a portion of which has threads, the shaft having a longitudinal axis, a distal end configured for guiding the rod into the U-shaped channel, a proximal end, a knob associated with the proximal end;
a sleeve defining a longitudinal axis and having a through bore, the first member received in the through bore;
a second member having a first end and a second end, wherein the second end is configured to engage one of the side walls to secure the instrument to the fastener such that the instrument is disposed outside the passageway, the second member pivotally engaged with the sleeve for positioning the distal end of the first member with respect to the first end of the second member; and
wherein rotation of the knob causes the distal end of the first member to move linearly along the direction of the longitudinal axis of the first member with respect to the sleeve for introducing the spinal rod into the U-shaped channel.

51. The instrument of claim 50, wherein the knob and the first member define a continuous channel extending from the proximal end to the distal end for receiving additional surgical instruments.

52. The instrument of claim 50, wherein each of the side walls of the bone fastener include a first and second end face, each first and second end face defining a recess;
wherein further the second end of the second member includes a pair of jaws, the jaws having a first extension and a second extension, the second extension located opposite the first extension, wherein further the first extension is configured to engage the recess of the first end face of one of the side walls and the second extension is configured to engage the recess of the second end face of the side wall.

53. The instrument of claim 52, wherein each of the recesses defines opposing end walls and a seat therebetween, the seats forming an angle with respect to a longitudinal axis defined by the U-shaped channel;
wherein further the first and second extension are dimensioned and configured so as to form a secure engagement with the opposing end walls and the seat to prevent rotation of the fastener with respect to the jaws.

54. The instrument of claim 52, wherein the angle is about 15° to about 90°.

55. The instrument of claim 52, wherein the angle is about 45° to about 75°.

56. The instrument of claim 52, wherein the angle is about 55° to about 60°.

57. The instrument of claim 50, wherein the side walls of the bone fastener include an exterior side surface, and the jaws define an interior surface configured for mating engagement with the exterior side surface to prevent rotation of the bone fastener with respect to the jaws.

58. The instrument of claim 57, wherein the side walls and the exterior side surface are substantially arcuate, and wherein further the interior surface of the jaws is substantially arcuate for secure engagement with the exterior side surface.

59. The instrument of claim 50, wherein the first end of the second member includes a pair of pivoting arms for bringing the second end of the second member into engagement with the side wall of the bone fastener.

60. The instrument of claim 59, wherein the pair of arms are resiliently biased open.

61. The instrument of claim 59, wherein the first end of the second member includes a screw and a nut threadably disposed about the screw for selectively fixing the distance between the pair of arms.

62. The instrument of claim 50, wherein the second member includes two parallel brackets disposed between the first end and the second end, wherein the brackets are configured for pivotal engagement with the sleeve, the sleeve disposed between the brackets such that the distal end of the first member is pivotable with respect to the first end of the second member.

63. The instrument of claim 50, wherein the first member includes a rod engaging tip disposed about the distal end of the first member for guiding the rod into the U-shaped channel, the rod engaging tip having a threaded central chamber for threaded engagement with the distal end of the first member and configured so as to be non-rotatable with respect to the sleeve, such that rotation of the first member causes the rod engaging tip to move linearly with respect to the sleeve and the first member.

64. The instrument of claim 63, wherein the rod engaging tip includes two parallel arches configured for secure engagement about the rod.

65. The instrument of claim 63, wherein rotation of the knob causes the rod engaging tip to move with respect to the first end of the second member.

66. A system for introducing a spinal rod, the system comprising:
a bone fastener having;
a head, the head including a top end, a bottom end;
a pair of side walls defining a top opening in the top end and an elongated U-shaped channel having a first longitudinal axis, the top opening and the channel further defining a passageway configured for receiving the spinal rod, each side wall further defining a first end face, a second end face, and an exterior side surface therebetween, the exterior side surface having a cavity; and
a bone engaging element extending from the bottom end of the head for anchoring the bone fastener to the bone;
the system further comprising a first tool for handling and manipulating the bone fastener, the first tool including:
a first member having first and second ends, the second end having a pair of jaws for securing the tool to one of the side walls of the head of the bone fastener such that second member is disposed outside of the passageway, and
the system further comprising at least a second tool for introducing the spinal rod into the U-shaped channel, the second tool comprising:
a shaft having a distal end and a proximal end;
a handle associated with the proximal end;
a prong associated with the distal end, the prong having a first leg and a second leg, the second leg spaced from and parallel to the first leg, the first and second legs dimensioned and configured to accommodate the head with the spinal rod proximate the top opening of the bone fastener between the first and second leg, the handle is substantially aligned with the passageway of the spinal rod; and
wherein the first and second legs each include an extension, the extensions dimensioned and configured for cammed engagement with the cavities of the exterior side surfaces so as to apply a pivoting force to the rod to introduce the rod into the U-shaped channel.

67. An instrument for manipulating a bone fastener having a pair of side walls defining a U-shaped channel having a top opening, the U-shaped channel and top opening defining a passageway configured for receiving a spinal rod, the instrument comprising:
an elongated member having a distal end and a proximal end; the distal end defining a pair of jaws configured for securing about one of the side walls of the fastener such that the instrument is disposed substantially outside the passageway;
a handle associated with the proximal end;
a shaft having a through bore, the shaft associated with the distal end of the elongated member;
wherein the shaft has a first position where the jaws are resiliently biased open and a second position where the jaws are drawn together in a closed position to secure about the side wall of the head of the fastener.

68. The instrument of claim 67, wherein each of the side walls include a first and second end face, each first and second end face defining a recess, the jaws having a first extension and a second extension, the second extension located opposite the first extension, wherein further the first extension is configured to engage the recess of the first end face and the second extension is configured to engage the recess of the second end face.

69. The instrument of claim 68, wherein the recess defines opposing end walls and a seat therebetween, the seat forming an included angle with respect to a longitudinal axis defined by the U-shaped channel;
wherein further the first and second extensions are dimensioned and configured so as to form a secure engagement with the opposing end walls and the seat to prevent rotation of the fastener with respect to the jaws.

70. The instrument of claim 68, wherein the angle is about 15° to about 90°.

71. The instrument of claim 68, wherein the angle is about 45° to about 75°.

72. The instrument of claim 68, wherein the angle is about 55° to about 60°.

73. The instrument of claim 67, wherein the side walls include an exterior side surface, and the jaws defining an interior surface configured for mating engagement with the exterior side surface to prevent rotation of the bone fastener with respect to the jaws in at least one direction.

74. The instrument of claim 73, wherein the side walls and the exterior side surface are substantially arcuate, and wherein further the interior surface of the jaws is substantially arcuate for secure engagement with the exterior side surface.

75. The instrument of claim 67, wherein the shaft slides with respect to the elongated member.

76. The instrument of claim 67, wherein at least a portion of the jaws is received in the through bore of the shaft to draw the jaws together in a closed position when the shaft is in the second position.

77. An instrument for manipulating a bone fastener having a head including a pair of side walls defining a U-shaped channel having a top opening, the U-shaped channel and top opening defining a passageway configured for receiving a spinal rod, the instrument comprising:
an elongated member having a distal end and a proximal end;
the distal end defining a pair of pivotable jaws configured for securing about one of the side walls of the fastener such that the instrument is disposed substantially outside the passageway;
the proximal end having a pair of forceps handles for pivoting the jaws, the forceps handles having a first position where the jaws are configured in a closed position for securing about the side wall and at least a second position where the jaws are configured in an open position.

78. The instrument of claim 77, wherein each of the side walls include a first and second end face, each first and second end face defining a recess, the jaws having a first extension and a second extension, the second extension located opposite the first extension, wherein further the first extension is configured to engage the recess of the first end face and the second extension is configured to engage the recess of the second end face.

79. The instrument of claim 78, wherein the recess defines opposing end walls and a seat therebetween, the seat forming an included angle with respect to a longitudinal axis defined by the U-shaped channel;
wherein further the first and second extensions are dimensioned and configured so as to form a secure engagement with the opposing end walls and the seat to prevent rotation of the fastener with respect to the jaws.

80. The instrument of claim 78, wherein the angle is about 15° to about 90°.

81. The instrument of claim 78, wherein the angle is about 45° to about 75°.

82. The instrument of claim 78, wherein the angle is about 55° to about 60°.

83. The instrument of claim 77, wherein the side walls include an exterior side surface, and the jaws defining an interior surface configured for mating engagement with the exterior side surface to prevent rotation of the bone fastener with respect to the jaws.

84. The instrument of claim 83, wherein the side walls and the exterior side surface are substantially arcuate, and wherein further the interior surface of the jaws is substantially arcuate for secure engagement with the exterior side surface.

85. The instrument of claim 77, wherein a ratchet mechanism is associated with the forceps handles, the ratchet mechanism configured such that the forceps handles may be selectively fixedly positioned in the first or at least second position.

86. An instrument for manipulating a bone fastener having a pair of side walls defining a U-shaped channel having a top opening, the U-shaped channel and top opening defining a passageway configured for receiving a spinal rod, the side walls including an exterior side surface having a cavity on the exterior side surface, the instrument comprising:
an elongated member having a distal end and a proximal end;
the distal end defining a pair of pivotable jaws configured for securing to the pair of side walls of the fastener such that the instrument is substantially aligned with the passageway, the jaws defining an interior surface including a first extension and a second extension each dimensioned and configured for pivotable engagement with the cavity, the second extension opposite the first extension; and
the proximal end having a pair of forceps handles for pivoting the jaws, the forceps handles having a first position where the jaws are configured in a closed position such that the first extension is engageable with the cavity of one of the side walls and the second extension is engageable with the cavity of the other side wall, and at least a second position where the jaws are configured in an open position.

87. The instrument of claim 86, wherein the proximal end is substantially arcuate with respect to a portion of the elongated member located between the proximal and distal end.

88. The instrument of claim 86, wherein the interior surface of the jaws defines a chamber configured for secure engagement with at least a portion of the exterior side surfaces of the side walls, the chamber being substantially circular cylindrical.
